(19) United States
(12) Patent Application Publication
(10) Pub. No.: US 2009/0148502 A1
(43) Pub. Date: Jun. 11, 2009

(54) COMPOSITIONS AND METHODS FOR TREATING LACERATIONS, ABRASIONS, AVULSIONS, BURNS, ULCERS, AND CASES OF EXCESSIVE BLEEDING

(75) Inventor: Allan D. Pronovost, San Diego, CA (US)

Correspondence Address: FOLEY & LARDNER LLP, 111 HUNTINGTON AVENUE, 26TH FLOOR, BOSTON, MA, 02199-7610, US

(73) Assignee: Hemo Nanoscience, LLC

(21) Appl. No.: 11/876,343

(22) Filed: Oct. 22, 2007

Related U.S. Application Data
(60) Provisional application No. 60/853,621, filed on Oct. 23, 2006.

(51) Int. Cl. A61K 9/70; A61K 9/14; A61K 31/28; A61K 38/02; A61K 33/00

(52) U.S. Cl. 424/447; 424/489; 424/501; 514/184; 514/2; 424/724

(57) ABSTRACT

Described herein are compositions and methods related to wound treatment. Compositions are multi-components admixed in amounts and ratios to meet specific objectives for optimally treating various types of wound injury.

### CROSS-REFERENCE TO RELATED PATENT APPLICATIONS

The present application claims the benefit of U.S. Provisional Application 60/853,621, filed Oct. 23, 2006.

### FIELD OF THE INVENTION

This invention relates to compositions and methods useful in treating wounds. In particular, the invention relates to compositions that stop excessive bleeding and methods for using the compositions.

### BACKGROUND OF THE INVENTION

A variety of wound-care and hemostatic products have been developed throughout history. The design requirements of any given formulation are largely driven by the specific needs of the type of wound, the availability and cost of materials and equipment, and the accessibility of the patient to professional medical care. For example, an individual who trips on the sidewalk and scrapes his or her knee has markedly different needs than a soldier in the battlefield experiencing severe arterial hemorrhage, who may be hours from the nearest emergency medical station. Likewise, while the application of human recombinant clotting factors may be partially effective in stopping bleeding, the cost of producing them is prohibitive to the general consumer or medical markets—hence the popularity of purely mechanical methods, e.g., the use of gauze and manually applied pressure to the wound site or the use of plastic adhesive bandages.

For treating excessive bleeding, no perfect solution currently exists. Heat generation with respect to one type of hemostatic agent is a major problem. The dressing's ability to adhere effectively when applied to deep wounds, or wounds of irregular shape, creates a major limitation. Sometimes cleansing a hemostatic agent from the wound can be a problem.

For sealing wounds and stopping minor bleeding, wound sealant agents have been in use for years, in many forms, with varying degrees of suitability to various classes of wounds. Conventional wound sealants fail to present an optimized combination of speed of clotting and are not effective under pressure bleeding conditions. Wound sealants for hemostatic control are typically 2 or 3 step multi-component formulations mixed prior to use and allowed to set before application. Wound sealants formulations of materials purified from human or animal blood or tissue products are typically slow to react, often requiring more than 30 minutes, and generally ineffective against pressure bleeding or recurrent bleeding.

Each new dressing material and each new technique show promise but no new material or technique has satisfied the need to significantly improve wound healing for patients with, for example, decubitus ulcers, pressure ulcers, burns, and other types of chronic wounds.

### SUMMARY OF THE INVENTION

The present invention provides multi-component multi-functional wound sealant compositions and methods to reduce, control, seal and/or eliminate bleeding or serous fluid exudation from minor lacerations, abrasions, avulsions, cuts, scrapes, scratches, burns, sunburns, ulcers, external vascular sites, internal vascular sites, and deep wound trauma, and to protect such wound sites from further environmental insult. The present invention can be delivered to the wound site as a solid powder, a liquid, gel, semi-gel, aerosol, or patch composite bandage, and may additionally deliver analgesic, antiseptic, and/or skin healing promoting activity to the wound. This invention also relates to compositions and methods for control and management of bleeding during and post surgery. This invention brings innovative solutions to reduce death caused by uncontrolled internal or external hemorrhage from heavy trauma and/or battlefield wounds.

Wound-care products should be multifunctional and control bleeding, protect against bacterial infection or contamination, control pain at the wound site, provide for adequate sealing or closure, protect wounds from the environment, and improve the healing process.

The present invention provides multifunctionality with an array of wound treatment products. Each of the products is composed of multi-components admixed in amounts and ratios to meet specific objectives for optimally treating different types of wound injuries. The primary binding agent comprises reactive submicron colloidal silica particles that, when hydrated, agglomerate in the form of a supra-molecular, cross-linked network serving as the base scaffolding component as the structural basis for a synthetic non-fibrin clot. The colloidal silica particles are nanometers in size and comprise a high density of specific, functionally-reactive surface hydroxyls to afford rapid scaffolding in situ. Multiple inorganic, inert, and optionally biologically active components are admixed with the nanoparticulate silica and each admixture is formulated to optimize product features to treat specific wound trauma. Each of the final products is formulated to be delivered to the wound site as a one-step single-delivery system. Applicable types of wounds include, for example, acute wounds, surgical wounds/incisions, traumatic wounds, lacerations, abrasions, contusions, pressure ulcers, venous insufficiency ulcers, arterial ulcers, neuropathic ulcers, diabetic ulcers, and burn wounds.

In one embodiment, the invention is directed to a wound management composition comprising: a preparation of silicon dioxide particles having a particulate diameter of 10 nm (range 0.5 nm to 200 nm), comprising a surface area up to 500 square meters per gram, and further having an average of four hydroxyl groups per square nanometer.

In a particular embodiment, the wound management composition can comprise a fluid removal agent selected from the group consisting of: ceramics, alumina or alumina silicate, silica or alumina gel, ceramic sorbent powder cationic exchanger, synthetic zeolyte Y powder, cross-linked polyamine, polyDADMAC, polyacrylamide, sodium polyacrylic acid, lignosulfates, silicaeous perlite, vermiculite, porous non-activated or activated carbon and hyaluron.

In a particular embodiment, the wound management composition can comprise an adhesion and/or clumping agent selected from the group consisting of: starch copolymers, poly 2-propenamide-co-2-propenoic acid including sodium or potassium salts thereof, bentonite clay, sodium or calcium bentonite, montmorillonite clay, smectite clay and magnesium lithium phyllosilicate.

In a particular embodiment, the wound management composition can comprise a thickening and/or swelling agent selected from the group consisting of: smectite clay, montmorillonite clay, sodium and calcium bentonite powder, aluminum oxide, magnesium aluminum silicates and silica gels and sodium polyacrylic acid, and starch copolymers, poly 2-propenamide-co-2-propenoic acid including sodium or potassium salts thereof which also help in adhesion and/or clumping.

In a particular embodiment, the wound management composition can comprise a delivery agent selected from the group consisting of allyl methacrylate cross polymers, cross-linked agarose gels, or other natural or synthetic drug delivery vehicles. The delivery agent can be loaded to deliver drugs or therapeutic agents such as anti-infectives, analgesics, astringents, anti-inflammatory agents or the like on a continuous release basis.

In a particular embodiment, the wound management composition can comprise an anti-infective agent selected from the group consisting of: silver sulfadiazine, neomycin triple antibiotic, vancomycin, silver nitrate (silver ions), 8-hydroxyquinoline and other anti-infectives, microstatic agents, or antiseptics such as Kathon, Neolone, or PVP-Iodine.

In a particular embodiment, the wound management composition can comprise an analgesic agent selected from the group consisting of: acetylated/non-acetylated salicylates, ibuprofen, diclofenac, naprosyn, piroxicam, difunisal, oxaprozin, sulindac, tolmetin sodium, nabumetone, mefanamic acid, fulurbiprofen, fenoprofen, meloxicam, meclofenemate, etodolac, ketoprofen, indomethacin, menthol, camphor, ethyl chloride, lidocaine, prilocaine, benzocaine, butacaine, cyclomethycaine, dibucaine, tetracaine, daspaicin, opioid analgesics and morphine and its derivatives.

In a particular embodiment, the wound management composition can comprise a cytokine selected from the group consisting of: platelet derived growth factor, granulocyte colony stimulating factor, fibroblast growth factor and epidermal growth factor.

In a particular embodiment, the wound management composition can comprise a thrombolytic cascade accelerant selected from the group consisting of: polyethylene glycol 3350, polyoxyethelene-6-sorbitol, non-ionic surfactants, polysorbate 60, polypeptide clotting factors, prothrombin, thrombin, thromboplastin and active fragments thereof. In another embodiment, the clotting factors are recombinant polypeptides, e.g., human polypeptides.

In a particular embodiment, the wound management composition can comprise a mordant selected from the group consisting of: cross-linked anionic or cationic polyamine or polyacrylamide flocculent material (PALMS), lignosulfanates, hyaluronan, synthetic polyketides, polyhydroxyalkanoates, cutin or suberin digests of plant material, naturally occurring polyesters, poly(g-D-glutamate), polymerized human serum albumin, bioplastic polymers, pullanan, scleroglucan, naturally occurring non-edible polysaccharides, dextran, polypeptide polymers, collagen and fibrinogen. Other common mordants include: guar gums, xanthum gums, cellulose, carboxy methyl cellulose, soluble or insoluble fiber, alginate, agar, agarose and starch.

In another embodiment, an oxygen source, e.g., hydrogen peroxide. In another embodiment, the wound management system can include cations such as calcium.

In a particular embodiment, the wound management composition is in a formulation selected from the group consisting of: a liquid, a coating on a bandage or patch, a foam, an aerosol, a gel and a semi-gel.

In one embodiment, the invention is directed to a method of making a wound sealant composition, comprising: a) obtaining a preparation of silicon dioxide particles having a particulate diameter of 10 nm (range 0.5 nm to 200 nm), comprising a surface area up to 500 square meters per gram; b) hydroxylating the silicon dioxide particles to an average of four hydroxyl groups per square nanometer; c) forming an admixture of the hydroxylated silicon dioxide particles with one or more agents selected from the group consisting of: a fluid removal agent, an adhesion agent, a thickening agent, a delivery agent, an anti-infective agent, an analgesic agent, a thrombolytic cascade accelerant, a mordant, monovalent or divalent cations and an oxygen source thereby forming a wound sealant composition. The wound composition can optionally be sterilized, if required. In a particular embodiment, the method can comprise admixing the wound sealant composition with an excipient, a surfactant, or a resin. In another embodiment, the method can comprise conjugating a polypeptide clotting agent or fragment thereof, to the hydroxylated silicon dioxide particles.

In another embodiment, the present invention is directed to a method of treating a wound comprising identifying a subject having a wound characterized by excessive bleeding, and administering to the subject a wound sealant composition described herein, thereby reducing or ameliorating the excessive bleeding. The wound can be, for example, an acute wound, a surgical wounds/incision, a traumatic wound, a penetration wound, a laceration, an abrasion, a contusion, a severed limb, a pressure ulcer, a venous insufficiency ulcer, an arterial ulcer, a neuropathic ulcer, a diabetic ulcer, and a burn wound.

In another embodiment, the present invention is directed to a method for inducing blood coagulation in a subject, comprising administering a hemostatic formulation comprising fumed silica. In a particular embodiment, the hemostatic formulation is a powder, e.g., sorbent powder. In another embodiment, the subject is treated with one or more anticoagulants, e.g., aspirin.

This invention is directed to various compositions formulated as a powder, liquid, gel, semi-gel, aerosol, or patch composite bandage that can be applied to skin surfaces and aids in stopping or controlling bleeding, forms a barrier to external contamination, and promotes wound healing while protecting against further injury the skin area that might result in renewed bleeding, sores, galling, blisters, or raw spots.

### DETAILED DESCRIPTION

The present invention provides multi-component multi-functional wound sealant compositions and methods to reduce, control, seal and/or eliminate bleeding or serous fluid exudation from minor lacerations, abrasions, avulsions, cuts, scrapes, scratches, burns, sunburns, ulcers, external vascular sites, internal vascular sites, and deep wound trauma, and to protect such wound sites from further environmental insult. The present invention can be delivered to the wound site as a solid powder, a liquid, gel, semi-gel, aerosol, or patch composite bandage, and may additionally deliver analgesic, antiseptic, and/or skin healing promoting activity to the wound. This invention also relates to compositions and methods for control and management of bleeding during and post surgery. This invention brings innovative solutions to reduce death caused by uncontrolled internal or external hemorrhage from heavy trauma and/or battlefield wounds.

An ideal wound sealant is a “true” one-step formulation and delivery process that is preferably composed of non-bioactive materials that are inert and GRAS; is a single-step procedure that requires substantially no extra preparation time (such as pre-wetting, mixing or activating); is instantly reactive and effective; is highly hydrophilic; augments and accelerates natural clotting processes; utilizes materials provided by the body at the wound site in response to amount and type of bleeding; controls large volume bleeding; can swell upon rehydration affording pressure within wound sites; controls immediate and sustained bleeding; provides a lattice web formation in situ after application that is based on the agents reactive properties; serves as a dynamic pliable and malleable wound dressing; is stable with a long shelf life; be equally effective regardless of the patient's blood type or individual ability to induce coagulation on their own; and whose composition can be modified to address the specific needs of different types of wounds, said variations in composition involving adjustment of the amount and type of multiple components used in the admixture, said selection be determined by functional need.

Active features of the ideal hemostatic wound-care product include beings composed of non-bioactive inert materials that are GRAS; capable of being applied in a single-step procedure with no extra preparation time; capable of instantly reactive and effective; controls large volume bleeding; controls immediate and sustained bleeding; augments and accelerates natural clotting processes; controls trauma pain; swells to provide pressure within wound sites; provides a lattice web scaffolding in situ; produces a dynamic pliable and malleable wound dressing; is stable with a long shelf life; is provided sterile, if required; is equally effective regardless of the patient's blood type or individual ability to induce coagulation on their own; and whose composition can be modified to address the specific needs of different types of wounds, said variations in composition involving adjustment of the amount and type of multiple components used in the admixture, said selection be determined by functional need.

Active features of the ideal surgical and emergency wound-care product include being composed of non-bioactive inert materials that are GRAS; capable of being applied in a single-step procedure with no extra preparation time; instantly reactive and effective; controls large volume bleeding; controls immediate and sustained bleeding; augments and accelerates natural clotting processes; controls trauma pain; swells to provide pressure within wound sites; provides a lattice web scaffolding in situ; produces a dynamic pliable and malleable wound dressing; is stable with a long shelf life; is provided sterile, if required; is equally effective regardless of the patient's blood type or individual ability to induce coagulation on their own; and whose composition can be modified to address the specific needs of different types of wounds, said variations in composition involving adjustment of the amount and type of multiple components used in the admixture, said selection be determined by functional need.

Active features of the ideal burn-injury wound-care product include providing a thin hydrogel layer on the surface of the skin; providing an external dry to the touch skin; promoting healing; minimizing scarring due to avoidance of skin grafts; promoting smooth healing; can be kept on for weeks; is stable with a long shelf life; can be removed without pain; holds moisture but does not participate in skin adhesion; and whose composition can be modified to address the specific needs of different types of wounds, said variations in composition involving adjustment of the amount and type of multiple components used in the admixture, said selection be determined by functional need.

Active features on the ideal decubitus ulcer, pressure ulcer, chronic wound product include provides protective and moist environment for wound healing; provides a dry to touch skin; provides durable seal; abrasion resistant seal; is stable with a long shelf life; promotes healing; and whose composition can be modified to address the specific needs of different types of wounds, said variations in composition involving adjustment of the amount and type of multiple components used in the admixture, said selection be determined by functional need.

Active features of the ideal over-the-counter (OTC) wound-care product include provide protective covering for minor lacerations and abrasions, avulsions, friction blisters, hangnails, finger cracks, and paper cuts; promote healing and create a moist wound environment; stop bleeding; keep out water, dirt and germs; provide a vehicle for delivery of medicaments; easy to apply and equally effective as a powder, liquid, gel, semi-gel, aerosol, or patch composite bandage; be equally effective regardless of the patient's blood type or individual ability to induce coagulation on their own; convenient to use; available in both unit-dosed (for first-aid kits) or multiple-use (for households) configurations; non-toxic and non-irritating to skin; is stable with a long shelf life; inexpensive; and whose composition can be modified to address the needs of companion animals.

Five areas of interest can be considered and incorporated into the design of each individual wound treatment product—the nature of the wound application to be treated, the wound treatment features needed to be addressed, the matrix of material components from which to design the specific wound treatment product, the system for delivering product to the specific wound site, and the formulation of the final product.

In one embodiment, the invention is directed to a process that optimizes the features of the final product to address the specific needs for treating the specific wound trauma. As such, materials are admixed in amounts and ratios to produce the final product that has the appropriate primary characteristics to address the primary treatment objectives. For example, an objective during hemorrhage is to stop blood loss; whereas, for burn injury, an objective is to protect the wound from environmental contamination. A product formulated as a powder can be better suited for treating hemorrhage, whereas a soft gel formulation can be more suitable for treating burns. Different product compositions with different product features and different delivery formulations are needed to optimally treat these two examples.

In another embodiment, the present invention is directed to a family of wound sealants that exploit high surface area, said high surface area having chemical functional groups of high density to allow for high hydrophilicity in addition to the use of said nanostructures to create lattice structures in situ especially in situations wherein natural clotting is impaired either naturally or through use of anticoagulant drugs, stimulate processes, and create additive opportunities, all to improve and accelerate blood clotting processes beyond the capabilities of prior art materials and methods.

The multi-component chemical functional group enriched nanostructure based wound sealant agent is formulated either as a non-sterile or sterile preparation for single-delivery application to a wound site, or as a multi-use preparation for household needs. The preparation can be packaged and supplied in several preferential formulations including: dry powder, dry adhesive coating, dry aerosol, semi-gel, gel, or liquid (non-aqueous). The formulations are applied topically to a wound site. Alternatively or in addition, the formulation can be introduced internally into the wound site in the case of, for example, deeper lacerations, arterial wounds, or during surgical procedures.

Depending on the functional need, major active features of the current invention can include 1) to avoid life-threatening exsanguination or hemorrhage; 2) to stabilize a patient's vital functions until surgical care is undertaken; 3) to extend resuscitation time to surgery by attenuating the effect of blood loss and shock; 4) to stop blood oozing for improved surgical control; 5) to promote blood coagulation; 6) to provide a protective moist wound healing environment; 7) to promote natural wound healing processes; 8) to staunch hemorrhage; 9) to control wound trauma pain; 10) to provide oxygen for promoting wound healing; 11) to provide protective “substitute skin” for large areas of ulcer and burn injury; 12) to protect against external contaminants; 13) to provide pressure at the wound site through rapid swelling of the composition so as to facilitate vessel closure; and 14) to protective against microbial infective agents.

The multi-component wound sealant according to the present invention uses clot accelerant lattice technology comprised of reactive hydrophilic non-porous silica nanoparticles; and in addition and optionally a fluid removal agent; and in addition and optionally an adhesion agent which may afford the additional property of rapid swelling for vessel closure; and in addition and optionally a thickening agent; and in addition and optionally cations; and in addition and optionally a delivery vehicle formulated as a powder, liquid, gel, semi-gel, aerosol, or patch composite bandage; and in addition and optionally medicaments and activators for treating the injury site; and in addition and optionally activators for enhancing the healing process; and in addition and optionally genetically engineered thrombin and thromboplastin by recombinant cloning.

The basic clot accelerant is short chains of non-porous silica nanoparticles that contain a very high density of highly reactive, hydrophilic surface hydroxyl groups. The high density of functional groups is achieved by high temperature hydrolysis (1800° C.) of chlorosilanes in a hydrogen oxygen flame to produce uniform nanometer size colloidal silica dioxide particles with a very high density of reactive hydroxyls. Upon contact with aqueous fluid at the wound site, these short chains of highly reactive nanoparticles instantly cross-link by hydrogen bonding with water. This lattice formed in situ is independent of fibrin and serves as a structured backbone for natural clot formation. The basic silica nanoparticles binding agent promotes rapid clot formation upon contact with aqueous fluid at the wound site by instantly cross-linking to form a hydrogen-bonded lattice. The silica nanoparticles form an intermediary in the lattice through hydrogen bonding with each other, or with polar water molecules. The binding agent assembled as a hydrogen bonded lattice becomes integrated throughout the wound, forming a barrier to blood loss but not impeding the function of the subject's intrinsic clotting factors supplied and activated by the bleeding itself. Indeed, the function provided by the silica lattice is to concentrate the subject's intrinsic clotting factors at the wound site by providing a synthetic scaffold hence trapping the cellular blood components and preventing further blood loss, and to serve as a temporary synthetic clot until the body is able to seal the wound by its own accord. This function is particularly valuable for patients treated with blood thinners or anticoagulants such as heparin, etc. Furthermore, the function provided by the silica lattice is independent of any types of medications the person may be administered, making it a powerful tool during surgical procedures.

The present invention provides a one-step single-delivery of a multiple-component wound sealant using silica nanoparticles that agglomerate into chains when hydrated by the aqueous component of blood, one result of which is thixotropy of the wound fluids. In addition, the compositions provide a one-step multi-component wound sealant that immediately seals and stops bleeding even to the capillary level, due to the nanometer dimensions of the non-porous agglomerated silica nanoparticles that crosslink from amorphous aggregates into a chemically cross-bonded three dimensional lattice. In addition, the compositions provide a one step multi-component wound sealant using nanoparticles of silica that is adaptable to a variety of single-delivery modes and media (dry, liquid, foam, gel, aerosol, and coating on patch/bandage), thus avoiding pre-wetting, pre-mixing, or activation time delay.

The present invention provides a wound sealant consisting of a network of silica nanoparticles, with all the advantages and features of the single-component/single-delivery sealant, to which materials and substances can be added to control bleeding and/or to enhance clotting. The compositions also provide a one step wound sealant comprising a network of non-porous silica nanoparticles to which various other clotting factors, calcium cations, astringents, accelerants, fibers, absorbent or adsorbent fluid removal agents, thickening swelling agents, adhesion and clumping agents, drug delivery vehicles for continuous release of drugs, in addition to antimicrobials, analgesics, anesthetics, mordants, and other components can be admixed to enhance clotting and optimize the material for different types of wounds, patients, environments, and hematological requirements.

The current invention provides a one-step multi-component wound sealant containing human recombinant thrombin and thromboplastin to accelerate the thrombolytic cascade in the case of deep wound or internal bleeding. In addition, the composition provides a wound sealant using nanoparticles of silica that is effective against serious trauma involving pressure (arterial) bleeding, not only to effectively stop the bleeding, but also to accommodate and utilize the clotting factors naturally present in the body fluid present at a wound site, without harming the native tissue or impeding further treatment by surgical or medical personnel.

The present invention provides a wound sealant that is an efficient transport vehicle for thrombolytic cascade accelerants and various clotting factors that can be incorporated as required into the tissue sealant formulation to facilitate control of pressure bleeding. The resulting compositions supply those thrombolytic cascade accelerant components naturally present in the body, as part of the thrombolytic cascade, that are found at relatively low, rate-limiting, serum concentration so as to accelerate rather than limit or scavenge the clotting process, versus those components found at relatively high concentrations and ready abundance in the serum. The present invention allows for the use of thrombolytic cascade accelerant precursor components as activators of the cascade at critical rate-limiting steps such as would occur with catalytic enzymatic processes. The compositions provide a wound sealant comprised of thrombolytic cascade accelerants supplied at greater-than physiological conditions.

In some embodiments, the present invention provides a family of wound sealants that exploit high surface area, highly hydrophilic non-porous silica nanoparticles (typically from fumed silica) to create lattice structures, stimulate processes, and create additive opportunities, all to improve and accelerate blood clotting process beyond the capabilities of prior art materials and methods. The compositions provide both a single-component/single-delivery wound sealant and a multi-component single-delivery wound sealant that, when hydrated, creates a fabric of agglomerated chains of silica nanoparticles in situ, to capture red cells and other blood components, impede their flow from the wound site, and thereby concentrate the subject's intrinsic clotting factors to accelerate clot formation. In the case of external bleeding wherein excess fluid is released, the present invention provides a one-step wound sealant consisting of silica nanoparticles dispersed with or coated onto other molecular water absorbents of larger particle size so as to keep the ratio of nanoparticles to fluid at the wound site within a certain ratio favoring lattice formation, viscosity, and degree of thickening. These absorbents work in tandem with the silica, removing excess water and serum from the wound site and further concentrating clotting factors to promote clot formation. Such absorbents may include inert materials of high water binding capacity such as silicaeous perlite or vermiculite, molecular sieve alumina or alumina silicate micro spheres, or alumina gels, ceramic microspheres, porous non-activated or activated carbon as absorbent, or the like. The compositions provide a wound sealant using nanoparticles of silica that provides sustained clotting at the application site due to the ability of the material to reform, permitting the handling of continuous or renewed bleeding.

In some embodiments, the present invention provides a wound sealant that is an efficient transport vehicle for thrombolytic cascade accelerants that is animal-derived or recombinant-derived in admixture with various admixtures of the multi-component wound sealant. The compositions provide an excess of critical core thrombolytic cascade accelerant precursor components involved in the activation of plasma components already supplied by the body as building blocks for clot formation versus supplying those essential clot formation dependent factors themselves (e.g., prothrombin or fibrinogen).

In some embodiments, the present invention provides a one-step, multiple-component wound sealant formulation that is not susceptible to self-activation or interaction between the components, while the formulation is in storage. Another object is to provide a one-step, multiple-component wound sealant that has a useful storage life, and that requires minimal special packaging and/or storage conditions. Another object is to provide a wound sealant using materials with cost-effectiveness superior to that of methods described in the prior art. Another object is to provide a wound sealant of nanoparticles of silica that uses inexpensive material from an inorganic source, thus reducing costs. The compositions use thrombolytic cascade accelerants composed of non-interactive components (not directly reactive with each other in the blood clotting process), such that the components can be formulated without concern for reaction or cross-reaction upon contact and formulation, and for which there is no need to keep components separate for fear of contact and “firing” the system. The present invention provides a wound sealant with thrombolytic cascade accelerant reagents that is stable, yet immediately bioactive in liquid form, including non-aqueous liquid formulations.

In some embodiments, the present invention includes a multi-component, wound-sealant composition having a crossing-bridging binding agent including a plurality of reactive silica nanoparticles having surface hydroxyl groups. The silica nanoparticles agglomerate into supramolecular lattice of hydrogen-bonded chains of silicon dioxide when applied to a bleeding wound. The silica nanoparticles have a surface area between about 25 square meters per gram and about 500 square meters per gram, between about 50 square meters per gram and about 400 square meters per gram, and between about 100 square meters per gram and about 250 square meters per gram. The silica nanoparticles have on average about four to eight hydroxyl groups per nanometers squared, within a range of between about 1 and about 20 hydroxyl groups per nanometers squared. The silica nanoparticles have an average diameter between about 0.1 nanometer and about 200 nanometers, between about 1 nanometer and about 80 nanometers, between about 5 nanometers and about 50 nanometers and between about 10 nanometers and about 40 nanometers.

In some embodiments, the present invention provides a multi-component, wound-sealant composition that includes, at least a cross-bridging binding agent of silica nanoparticles having surface hydroxyl groups (active component A), and none, one, or more than one additional components including a fluid removal agent (active component B), an adhesion/clumping agent (active component C), a thickening swelling agent (active component D), a drug delivery vehicle (active component E), clot-enhancing biological compositions (active component F), and an activator or accelerator (active component G).

In some embodiments, the multi-component, silica nanoparticle wound-sealant composition, when applied to a wound site, accelerates hemostasis at the wound site. At the wound site, the multi-component silica admixture dessicates the wound site upon contact and primary hydration; removes water and concentrates the blood; thickens and removes excess blood; reduces blood flow; agglomerates into a supramolecular lattice of hydrogen-bonded chains of silicon dioxide which tightens into a solid lattice upon full cross-bonding resulting in constriction and closure of vessels at the submicron level; rapidly swells and places pressure on vessels further constricting flow; forms an adhesive binding; and delivers clotting agent accelerants to accelerate hemostasis by activating the blood-clotting cascade.

In some embodiments, the invention provides a method of inhibiting bleeding in a mammal by applying an effective quantity of the multi-component, silica nanoparticle wound-sealant composition to inhibit the bleeding and to induce the clotting cascade to initiate hemostasis in the subject. The composition of this wound sealant is provided as a single dry powder admixture that is ready-to-use to inhibit bleeding. No additional mixing, preparation, or other manipulation is required for its use. Other wound sealant formulations include: a liquid; a coating on a bandage or patch; a foam; an aerosol; and a gel or semi-gel. In each formulation, the wound sealant product is applied as a single-step process and thus avoids pre-wetting, pre-mixing, or any activation step that would provide time delay to the otherwise immediate activation of the wound sealant's actions.

In one embodiment of the present invention, an inert powder of fumed silica nanoparticles and various other medicament agents are mixed with a solution of nitrocellulose to provide the basis for a liquid form of excellent adhesive skin binders and therapeutic constituents in solvent. Various admixtures of these constituents provide the desired properties for a wound protectant and medicament delivery system for treating minor abrasions, cuts, scrapes, scratches, burns, sunburns, ulcers and other skin injuries and irritations. Specifically, plastic (nitrocellulose) powder in a biocompatible, rapidly drying solvent is used to accomplish the desired results. The basic objectives are to provide a non-fiber matrix to both protect and medicate the site of injury.

The multi-component silica nanoparticle wound sealant is composed primarily of agents that are generally regarded as safe (GRAS) by the FDA and other regulatory agencies. The multi-component silica nanoparticle wound sealant system consists of bio-tolerable components chosen of the appropriate size and of the appropriate physical and chemical properties to be effective for reducing blood flow and for accelerating blood clotting. The base clot forming components are inorganic and inert.

The multi-component silica nanoparticle topical wound care product admixture in the form of a coating on a bandage or patch is applied by securing the bandage or patch over or into the site of injury, which can be internal or external. The particulate admixture of components on the bandage or patch is attracted to and restrained on or in a surface of a minor abrasion, cut, scrape, scratch, burn, sunburn, ulcer or other wound injury or irritation, and reduces and thickens free blood flow and excess blood in the site of the injury. A lattice is formed of the thickened blood and the silica nanoparticles. The adhesive binding agent in the multi-component topical wound care product reduces or stops further blood flow from the injury site.

An object of the present invention is to provide a powder or fluid composition adapted to form a protective or preventative covering or bandage for minor abrasions, cuts, scrapes, scratches, burns, sunburns, ulcers and other skin injuries and irritations, such as bleeding during and post-surgery, and uncontrolled internal and external hemorrhage from heavy trauma and/or battlefield wounds.

Another object of the present invention is to provide a powder or fluid composition adapted to form a seal on non-superficial tissues or to close open tissues exceeding minor abrasions, cuts, scrapes, scratches, burns, sunburns, ulcers and other skin injuries and irritations.

An object of the present invention is to provide a composition in the form of a powder liquid, gel, semi-gel, aerosol, or patch composite bandage.

An object of the present invention is to provide a powder or fluid composition formulated such that the combination of short and long alkyl chain monomers and/or plasticizers ensure flexibility of the resulting polymerized protective coating or seal.

An object of the present invention is to provide a powder or fluid composition formulated such that the consistency or viscosity may be optimized for the intended application.

An object of the present invention is to provide a powder or fluid composition formulated such that a stabilizer can be incorporated to enhance stability of the composition.

An object of the present invention is to provide a powder or fluid composition packaged such that multiple applications may be dispensed out of the same container.

An object of the present invention is to provide a power or fluid composition packaged such that a trial quantity or limited number of applications may be dispensed.

An objective of the present invention is to provide a powder or fluid composition comprised of a composition adapted to form, in situ, a protective superimposing element, seal or covering element that covers and or fills the site of injury.

Free-flowing powder embodiments of the multi-component silica nanoparticle wound sealant admixture are applied to the surface of a wound in the presence of liquid blood. As the free-flowing particulate sealant is restrained on or in a surface of a wound, blood flow is reduced and the excess blood in the wound area is thickened. A cross-linked lattice is formed of the thickened blood and the silica nanoparticles. The adhesive binding agent in the multi-component sealant when placed over the wound area or in the wound site reduces or stops further blood flow from the wound site. One or more clotting agent accelerants in the sealant admixture activate the blood-clotting cascade.

Liquid embodiments of the multi-component, silica nanoparticle wound-sealant admixture are applied by pouring the sealant over the surface of a wound in the presence of liquid blood. The liquid particulate sealant is attracted to and restrained on or in a surface of a wound, blood flow is reduced and the excess blood in the wound area is thickened. A cross-linked lattice is formed of the thickened blood and the silica nanoparticles. The adhesive binding agent in the multi-component sealant when placed the over the wound area reduces or stops further blood flow from the wound site. One or more clotting agent accelerants in the sealant admixture activate the blood-clotting cascade.

Embodiments of the multi-component, silica nanoparticle wound-sealant admixture in the form of a coating on a bandage or patch are applied by securing the bandage or patch over the surface of a wound in the presence of liquid blood. The particulate sealant on the bandage or patch is attracted to and restrained on or in a surface of a wound, blood flow is reduced, and the excess blood in the wound area is thickened. A cross-linked lattice is formed of the thickened blood and the silica nanoparticles. The adhesive binding agent in the multi-component sealant when placed over the wound area reduces or stops further blood flow from the wound site. One or more clotting-agent accelerants in the sealant admixture activate the blood-clotting cascade.

Embodiments of the multi-component, silica nanoparticle wound-sealant admixture in the form of a foam or an aerosol can be applied by spraying the wound sealant over the surface of a wound in the presence of liquid blood. The particulate sealant in the foam or aerosol is attracted to and restrained on or in a surface of a wound, blood flow is reduced and the excess blood in the wound area is thickened. A cross-linked lattice is formed of the thickened blood and the silica nanoparticles. The adhesive binding agent in the multi-component sealant when placed the over the wound area reduces or stops further blood flow from the wound site. One or more clotting-agent accelerants in the sealant admixture activate the blood-clotting cascade.

Embodiments of the multi-component, silica nanoparticle wound-sealant admixture in the form of a gel or semi-gel can be applied by placing and securing the gel or semi-gel over the surface of a wound in the presence of liquid blood. The particulate sealant is attracted to and restrained on or in a surface of a wound, blood flow is reduced, and the excess blood in the wound area is thickened. A cross-linked lattice is formed of the thickened blood and the silica nanoparticles. The adhesive binding agent in the multi-component sealant when placed the over the wound area reduces or stops further blood flow from the wound site. One or more clotting-agent accelerants in the sealant admixture activate the blood-clotting cascade.

The present invention provides a method of making a wound-sealant composition including an admixture of a plurality of silica nanoparticles, hydroxylating the silica nanoparticles, plus one or more of the following: fluid-removal agent particles; adhesion/clumping agent particles; thickening swelling-agent particles; delivery-agent components; and cations; and optionally sterilizing the admixture. To this multi-component admixture, one or more additional compounds (such as an excipient, a surfactant, a resin, an antibiotic, an absorbent, an enzyme involved in clotting pathways, an antifungal agent, an astringent, an antiseptic, an analgesic, an anesthetic, polyfunctional short-chain molecules, and a mordant) can be added to provide various embodiments of formulations. The invention includes conjugating a clotting agent to the sterilized hydroxylated silica nanoparticles.

### Matrix of Materials (MOM)

Active components comprising the multi-component sealant are selected from a list of materials and ingredients that have different active features. The active component or the major features of materials in this selection matrix included: A) cross-bridging agent; B) fluid removal agent; C) adhesion clumping agent; D) thickening/swelling agent; E) drug delivery vehicle; F) biological agents to accelerate blood-clotting; and G) activator or accelerator agents to “bring into balance” the effectiveness of the final multi-component formulation. Each wound-sealant product is composed of active Component A plus none, one, or more of Component B, Component C, Component D, Component E, Component F, and Component G.

Component Designations: |A|—Component A—cross-bridging binding agent; |B|—Component B—fluid-removal agent; |C|—Component C—adhesion or clumping agent; |D|—Component D—thickening agent; |E|—Component E—delivery vehicle for drugs, therapeutic agents and or cell factors to promote cell growth and wound site healing; |F|—Component F—biological material for accelerating blood clotting; |G|—Component G—activator or accelerator agents used to increase functional effectiveness.

The specific admixture of this multi component (as many as seven or more than seven) matrix-of-materials or “MOM” formulation is considered and designed separately and specifically for each application such as for treating hemorrhages, or burns, or bedsores, or intra-abdominal bleeding, cuts or scrapes, or any other type of wound trauma. The MOM formulations for each specific application are not identical and include different materials representing any or all of each of the separate MOM components, and or specific amounts of each of the seven components and/or ratios of those components.

The component concentrations and component ratios in the MOM formulation for a specific application consider, and are adjusted for, the physical form in which the product is being delivered to the wound site. Final formulations are specific for the type of wound and for the physical form of the final product.

The final MOM mixture to be formulated can be comprised of only one active cross-bridging agent |A| such as fumed silica. Or, the final MOM mixture for a different specific application can include materials from all seven groups of active components, depicted as |A|B|C|D|E|F|G|.

The active components for use in developing specific MOM formulations are available from multiple suppliers. The materials used or representing each active component, say component B or C, can vary as long as the functional properties of said component are achieved. The detailed characteristics of materials obtained from different suppliers are not identical. Each individual and specific MOM formulation is developed according to the specific amounts of each active material in the MOM to reflect the desired characteristics of the final formulated multi-component sealant product. A range of quantities defines each active matrix component in the MOM to allow for variability in starting materials.

The composition of each multi-component sealant are designed specifically and separately for its intended use. Each multi-component MOM formulation is designed specifically for each application and includes a designated quantity and ratio range for all the active components. More than one MOM formulation can be effective for a specific application. Each MOM is formulated to be delivered as a one-step, single-component sealant.

### Component Materials

The composition of each wound sealant formulation is derived from as many as seven, or more than seven, multiple active components.

### Component A

### Cross-Bridging/Binding Agent

The properties of the cross-bridging binding agent include: 1) hydrogen bonding of surface rich hydroxyl-groups to each other and through polar water, 2) short chains of 10-20 nm sized nanoparticles in untreated fumed silica, 3) cross-linking of silica short chains to produce a three-dimensional lattice, 4) ability of the chains to rearrange and re-crosslink into a new three-dimensional lattice configuration upon shear, 5) a highly hydrophilic powder that dessicates on contact upon primary hydration, 6) low moisture content, 7) amorphous, 8) absorbs water upon hydration but is insoluble, chemically inert, non-toxic, and GRAS, and 9) upon full chemical bonding and cross-linking, shrinks and tightens affording constriction of blood vessels in the wound site. In some embodiments, addition of up to 20% Alumina oxide may aid in thickening of aqueous solutions (maximum thickening enhancement has been observed at approximately 16% alumina oxide).

The untreated CAB-O-SIL fumed silica products commercially available from Cabot Corporation, or fumed silica, commercially available from Degussa, are fine white powders with a specific gravity of 2.2. They are typically 99.8% pure by weight SiO₂ and are very suitable for use in adhesives and sealants. The surface chemistry of CAB-O-SIL framed silica influences moisture content, reinforcement properties, and rheology control. The chemical groups on the surface of untreated CAB-O-SIL fumed silicates are the isolated silanol and hydrogen bonded silanol, which are both hydrophilic, and the inert siloxane group, which is hydrophobic. CAB-O-SIL fumed silica imparts viscosity build-up and flow control properties to polymer systems by forming a three dimensional interacting network of silica aggregates throughout the system. The aggregates interact with one another through the hydrogen bonding of their surface silanol groups, restricting the flow and increasing the viscosity of the system. If the polymer compound is stirred, many of the hydrogen bonds are broken; the system loses viscosity and it becomes easier to coat surfaces or extrude from a tube. As soon as the shearing force is removed, the hydrogen bonds begin to reform and the viscosity of the polymer system increases again resulting in cross-linking.

Additives can be used with CAB-O-SIL fumed silica to enhance the network through the formation of additional bridges. This can result in increased viscosity.

Cabot Corporation supplies a number of different fumed silica products suitable for use and includes various products with a large hydrophilic surface area: CAB-O-SIL EH-5 (380 meter squared per gram of surface area), CAB-O-SIL HS-5 (325 meter squared per gram of surface area), CAB-O-SIL M-5P (200 meter squared per gram of surface area), CAB-O-SIL M-5 (200 meter squared per gram of surface area), CAB-O-SIL PTG (200 meter squared per gram of surface area), CAB-O-SIL MS-55 (255 meter squared per gram of surface area), CAB-O-SIL LM-150 (160 meter squared per gram of surface area).

Degussa provides a series of AEROSIL fumed silica products that include: AEROSIL COX 84, VP AEROPERL 300 Pharma (300 meter squared per gram of surface area), AEROSIL 200 (200 meter squared per gram of surface area), AEROSIL 200 VV Pharma (200 meter squared per gram of surface area), AEROSIL MOX 170, and AEROSIL MOX 80.

The present invention provides a composition of hydroxylated non-porous silica nanoparticles that, when applied to a wound, site cross-bridge to form a hydrogen bonded clot-accelerant lattice. The hydroxylated non-porous silica nanoparticles are also referred to herein as binding agents, and the preparation can be applied directly to the wound site to staunch simple vascular bleeding (cuts and scrapes). Silica (silicon dioxide) particles that are small enough to have surface area as high as 500 square meters per gram and an average of four to eight (4-8) hydroxyl groups per nanometer squared, with a range between about 1 and about 20 hydroxyl groups per nanometer squared are generally nanometer in size. These nanoparticles are extremely small (from about 0.01 nanometers to about 1 micrometer in diameter). The small size, coupled with a large surface area, allows for an excessive number of reactive hydroxyl groups to facilitate cross linking in the highly polar water environment found in blood.

Silica nanoparticles are non-porous hydroxysilica nanoparticles that can be used as binding agents and are not to be confused with larger chemically-inert silica macro- or microparticles (greater than one micrometer in diameter), which are produced by grinding and sieving, and are commonly used in the food industry for anti-caking purposes. The conventional larger silica particles lack the necessary active hydroxyl functional groups on the surface of the particle.

For external bleeding applications, the binding agent is comprised of fumed silica nanoparticles in short chains with individual surface areas up to about 500 square meters per gram, and preferably with individual particle sizes as small as a few nanometers in diameter. Such silica particles are “fumed silica” produced by Cabot. Medical grade fumed silica for human use is relatively rare (e.g., Cabot sells CAB-O-SIL grades M5 or MSP suitable for human applications). For other applications where medical grade quality is not as critical (e.g., life threatening trauma or battlefield conditions), Cabot grades L-90, LM-130, LM-150, PTG, M-7D, MS-55, H-5, HS-5, or EH-5 can be used. All grades fall within the range of 90-380 M2/g average surface area, less than 0.02% 325 mesh residue (44 microns), a size less than 100 nanometers, and have appropriate reactive surface chemistry.

During the fuming process used to prepare the nanoparticles, numerous surface hydroxyl groups are produced on the surface of the particle, which renders the particles highly hydrophilic. Hydroxylated fumed silica is produced by condensation of silicon dioxide molecules that are synthesized by hydrolysis of silicon tetrachloride in a hydrogen oxygen flame at 1800° C. In some embodiments, the surface density of hydroxyl groups average four hydroxyls per square nanometer. The material, once produced, is sterile.

To facilitate appreciation of the preferred embodiment of this invention, certain of the characteristics and functions of fumed silica are as follows:
When hydrated the binding agent instantly agglomerates into a supramolecular network, or fabric, of cross-bridging chains of silicon dioxide, in a lattice form that provides a three dimensional scaffold for clot formation with dimensions below one micron. Such dimensions on the scale of red blood cells, platelets, and other clotting factors permit effectiveness at every level of bleeding, even down to the capillary level.
As the water present in the blood and serum at the wound site is absorbed by the silica, a three dimensional silica/water lattice is formed based on hydrogen bonding of nanoparticle to nanoparticle both directly and indirectly through water as an intermediary through the hydrogen atoms of the molecule, and blood flow from the wound site is reduced. The decrease in water content at the wound site resulting from lattice formation also serves to concentrate the body's natural clotting factors, thus accelerating the clotting cascade. In addition, the 3 dimensional labyrinth serves as the scaffolding to allow fibrin attachment and formation.
The three-dimensional nature of the silica lattice also causes the dispersion to become a thyrotrophic gel in the absence of sheer forces. Upon the application of sheer forces, hydrogen bonds are broken and the lattice again becomes a flexible wound dressing that continually reforms itself when said sheer forces are removed, provided additional bodily fluid is available at the wound site to enable cross-bridging of the particles,
Over time, the extent of hydrogen bonding increases in three dimensions throughout the matrix. As a result of wound desiccation and the continued hydrogen bonding over time, this excessive cross-linking serves to tighten the labyrinth into the most heavily cross-bonded form the structure can achieve in situ. This tightening serves to help form a very tight and durable scab that further helps to constrict and close ruptured blood vessels, entrap blood cells and provides a solid structural foundation for continued fibrin adherence and deposition. The resultant clot, once dry, is stronger than a natural clot and oftentimes the skin or tissue around the wound site is pulled together as evidence of such. In fact, the formulations need to be adjusted to control the shrinkage and tightness. This is readily accomplished through the other MOM additives.
While the non-porous fumed silica bridging/binding agent is itself a useful wound sealant, it is also a convenient non-interactive carrier of other components to enhance the clotting and wound-sealing processes.

The binding agent is therefore a stand-alone, single-component/single-delivery sealant comprised of silica particles, prepared as a sterile material. These particles are a few nanometers in diameter, and have surface groups of hydroxyls and siloxanes capable of hydrogen bonding at the site of application. Hydroxyl groups are also known to beneficially initiate irritation of platelet membranes in wounds with the subsequent release of required clotting factors. Free hydroxyl groups in a wound produce a sting reaction owing to the caustic alkali, but that effect is eliminated herein. In this formulation, however, the hydroxyl groups are found bound to the silica surface at high density and serve to attract and entrap platelets but do not produce the sting reaction at the wound site as is noted with certain oxyacid preparations that require addition of a cation exchange material to offset the sting reaction (see for example, U.S. Patent Publication No. US20020141964). This is viewed as a beneficial feature. Upon hydration by aqueous body fluids, the binding agent immediately creates a web formed through hydrogen bonding that both provides a matrix for clotting and thixotropically reduces flow of the aqueous component of the blood. In addition to wound fluid flow reduction, the platelets present at the lattice interface will be effectively induced by the mass excess of hydroxyls to release clotting factors and further accelerate the clotting cascade.

Silica can be used as long or short chains of agglomerated nanoparticles ranging in surface area from 25 square meters per gram to five-hundred square meters per gram or greater but more preferably about 200 square meters per gram. The degree of network formation is dependent upon several factors that can be controlled either through the formulation and compounding or in the method of application at time of use. The concentration and grade of nanoparticle influence three dimensional network formations. The grades and concentrations described herein have been found to work. The pH in the wound site is also important. A pH of greater than about 2.3 up to about 8 is suitable, preferably between about pH 5 to about pH 7. The isoelectric point for nanosilica is approximately 2.3 where it is electrically neutral. Most blood samples have pH values between 4 and 9. The degree of dispersion in a blood sample is also important. The high hydrophilicity of reactive silica nanoparticles for water in a wound site routinely assures the ‘draw-in’ of aqueous fluid into the admixture once applied as a powder to the skin. This assures adequate and rapid dispersion. The use of non-aqueous based liquid formulations is also effective, as aqueous fluid from the wound is drawn into the admixture as solvent evaporates from the skin surface above the wound site, assuring adequate dispersion.

### Component B

### Fluid-Removal Agent

The properties of the fluid removal agent include being ultra rapid and effective in the absorption of fluid. Preferably, the material has the property of being effective in the retention of fluid in an inner hollow core, without separation while being dispersed in the fluid media itself and with minimal but controlled back release. The material is also preferably a desiccant, highly hydrophilic powder with porous microspheres of an average diameter of about 20-35 microns. Other characteristics and properties of the fluid removal agent include having low moisture content, being amorphous, and having the ability to absorb water upon hydration. The pores of said microspheres are small enough to allow both fluid and electrolytes to enter. Pores of a size that allows small ions and electrolytes to enter is most suitable. Any fluid and electrolytes that enter the microspheres will be kept sterile within the microsphere. An important consideration, however, is the requirement that the microspheres do not bind calcium, which is necessary for clotting. This means the fluid removal agent is selective to cations that are exchanged. In addition, the material is preferably chemically inert, non-toxic, and GRAS. Another property of the fluid removal agent is that over time it must also serve as a humectant, to release sterile liquid back to the clot to keep it moist and pliable.

Materials that have these general properties include ceramics, alumina and silica gel compositions. Engelhard provides a suitable product—ATS, which is 100% active ceramic sorbent powder cationic exchanger with high affinity for lead and no affinity for Ca⁺², wherein Ca⁺² is necessary for effective natural clotting.

Dry, flocculent, neutral, anionic or cationic, cross-linked polyamine, polyDADMAC, or polyacrylamide (Cytec, Inc., SUPERFLOC) can be used for fluid absorption as well or to aid as a mordant and are available in a variety of molecular weights of varying viscosity. Lignosulfates are naturally occurring GRAS materials extracted from wood pulp by various processes and are used in animal feeds and as indirect food additives. They occur in polymeric form following digestion and are hydrophilic and are used as adhesives, binders and sequestrants. Hyaluron is a GRAS linear polysaccharide used in cosmetics.

### Component C

### Adhesion (Clumping) Agent

Properties of the adhesive, clumping agent includes immediate clumping and particle adhesion upon wetting. Preferably, the material is a binder and plasticizer that is highly hydrophilic and expands upon wetting (about 1:1 up to about 25:1). The clumping agents are a microfine powder of mesh size >200 microns with low moisture content. It is amorphous, absorbs water upon hydration and is insoluble in water. Further, it is chemically inert, naturally occurring, non-toxic, and GRAS.

The preferable adhesion agent to use is calcium bentonite clay as a powder, which swells and clumps less than sodium bentonite and does not ion exchange calcium. The use of sodium bentonite may require calcium addition to offset ion exchange. Neither material releases heat upon hydration.

Cimbar Performance Materials, Cartersville, Ga., provides SUSPENGEL bentonite clay products that provide the characteristics for an adhesion or clumping agent. The SUSPENGEL 325 PLUS product is a 325 mesh sodium bentonite clay product (44 microns) used as theological additive in which a small amount of an additive is present to allow the formulation to wet out faster. Cimbar's CAL-BEN is a 200 mesh (74 microns) calcium bentonite powder.

American Colloid Company (ACC), Arlington Heights, Ill. provides industrial specialty clays including Panther Creek Dry Processed Calcium Bentonite, which is a 200 mesh (74 micron) calcium bentonite powder (natural Montmorillonite clay). ACC also provides Hectalite GM, which is a white calcium hectorite powder of Smectite clay containing the natural Mg Li Phyllosilicate mineral with a swelling ratio of 5:1. Other useful adhesive agents that also absorb up to 900× their weight in liquid are sodium polyacrylic acid and starch copolymers such as, for example, poly 2-propenamide-co-2-propenoic acid including sodium or potassium salts thereof.

### Component D

### Thickening Swelling Agent

The properties to the thickening swelling agent include being a highly efficient and rapid thickener, an effective rheology modifier that produces very high viscosity (up to 2200 cps at 5% solids). The thickening agent is preferably very highly hydrophilic and expands upon absorption of water (from greater than 20:1 up to 900:1). These agents are microfine powder or granular in form with mesh size greater than 100 (149 microns), low moisture content, amorphous, and insoluble. In addition, thickening agents absorb water upon hydration, are chemically inert, are naturally occurring, are non-toxic, and are GRAS. Products, such as ACC/AMCOL/CETCO products, are all available irradiated.

Superabsorbent starch copolymers can be used as thickening agents, such as Waterlock superabsorbent starch copolymer commercially available from Grain Processing Corp. (GPC). Starch graft 20 mesh polymers Waterlock G-430 (swell rate 500 plus) and Waterlock G-400 (swell rate 600) are Superabsorbent Polymers composed of poly (2-propenamide-co-2-propenoic acid, sodium salt). Additional GPC Waterlock products provide superabsorbent starch graft copolymers of poly (2-propenamide-co-2-propenoic acid), which are available as sodium or potassium salt and include Waterlock A100 (20 mesh, swell rate 130-200 plus), Waterlock A180 (20 mesh, swell rate 120-200 plus), and Waterlock A220 (40-60 mesh, swell rate 300-350 plus).

Thickening agents can include industrial specialty clays, such as Bentobrite 770, a natural white sodium bentonite and Montmorillonite, a natural clay provided as a micronized powder (325 mesh, dry processed sodium and calcium bentonite), each commercially available from the American Colloid Company of Arlington Heights, Ill. The American Colloid Company also provides VOLCLAY 325 mesh and VOLCLAY HPM75 dry processed microfine sodium bentonite.

Additional agents include a highly purified pharmaceutical grade Magnesium Aluminum Silicates, such as MAGNABRITE HV (high viscosity), a selected blend of white smectite clays (Mg Al silicate mineral) that provides viscosity of 800-2200 cps at 5%, commercially available from AMCOL Health and Beauty Solutions, Inc. (AMCOL). AMCOL also provides highly purified white bentonites and functional hydrogels, Polargel Volclay NF-BC pharmaceutical grade, irradiated, and water washed. This product includes sodium and calcium bentonite, montmorillonite clay powder with a swelling power of 24 ml/gm. Additionally, AMCOL provides its highly purified white bentonites and functional hydrogels as Polargel IVP, which is water-washed, surface-modified sodium Montmorillonite clay plus organic polymer; INCI PVP intercalated, which is designed to build viscosity in polar aqueous solvents (325 mesh, powder). Super absorbent polymers, as used in diapers, are available as sodium salts of polyacrylic acid, co-polymerized with acrylamide and ethylenebis (acrylamide).

Other agents include, silica gel products composed of very highly adsorptive material, such as the silica gel products commercially available from Qingdao Makall Group Co. Ltd. (Makall), Qingdao, China or Zeochem AG. These products are amorphous substances that are insoluble in water and other solvents, are nontoxic, and are chemically stable (SiO₂.nH₂O). The various types of silica gels formulated by Makall and Zeochem have different pore structures with unique chemical compositions and physical structures. These products are distinguished with high adsorption features, stable thermal performance, stable physical properties, and relatively high mechanical strengths. Makall Silica Gel products are differentiated according to their pore diameters. Makall Narrow Pore Silica Gels (SG01/SG02) are described as comprised of bead sizes from 1.4 to 8.0 millimeters that contain an inner structure of pore volume 0.35-0.45 ml/g, pore diameter of 2 to 3 nanometers and surface area of greater than about 600 square meters per gram Makall Middle Pore Silica Gels (SG03/SG04) are described as comprised of bead sizes from 2.0 to 8.0 millimeters that contain an inner structure of pore volume 0.5-0.8 milliliters per gram, pore diameter of 5 to 8 nanometers and surface area of 450-600 square meters per gram. Makall Wide Pore Silica Gels (SG05/SG06) are described as composed of bead sizes from 14 to 8.0 millimeters that contain an inner structure of pore volume 0.78-0.1.00 milliliter per gram, pore diameter of 8 to 10 nanometers and surface area of 350-500 square meters per gram.

### Component E

### Delivery Vehicle

Various types of delivery vehicles may be envisioned, including but no limited to: dry, free-flowing powders; non-aqueous liquids, sprays, or aerosols; and dry or liquid materials with inherent physical properties, such as hydrophilicity, hydrophobicity, lipophilicity, amphiphilicity, negative or positive charges, or amphotericity. The delivery vehicle can serve two purposes, either as a means for controlled-release of encapsulated drug or therapeutic agents over time, or to enable one to apply the product to the wound site in a uniform, easily-dispensed manner that enables activation of any and all properties that are imparted by the specific admixture of choice. Variations on the delivery vehicle examples presented below will be apparent to one skilled in the art.

The properties of the multi-functional highly adsorbent controlled release polymers with high-intruded volume include the ability to simultaneously load hydrophilic and lipophilic actives as a delivery system. Included in the formulation are agents that provide controlled delivery of functional ingredients with secondary thickening. The delivery agent is highly hydrophilic (delivery agent and functional additive can be provided in a dry state in admixture) and effective in rehydration and absorption of fluid upon exposure to aqueous solutions. These delivery agents are effective in retention of fluid with functional actives in the inner core. Dispersed in the fluid media itself are porous, amorphous microsphere particles (20-35 microns average diameter) with low moisture content. These polymers adsorb functional additive, absorb water upon hydration and are chemically inert, non-toxic, and GRAS.

AMCOL provides a highly adsorptive polymer, PolyPore E200, which is an allyl-methacrylate copolymer available as a white free flowing powder (20 micron). This multi-functional adsorbent polymer helps to stabilize and protect sensitive ingredients from degradation. It is simultaneously both hydrophilic and lipophilic, thus enabling an almost endless range of delivery systems where it can be used to stabilize and protect biologically active materials or control the rate of delivery while targeting the site of action.

A number of anti-infectives, analgesics/NSAIDS, local anesthetics, and other actives are available for being added to the MOM formulation for delivery with the wound sealant. They can be added on a controlled release basis through use of appropriate microstructures such as PolyPore E200, or they can be added directly. Potential anti-infectives include silver sulfadiazine, Neomycin triple antibiotic, Vancomycin, silver nitrate (silver ions), 8-hydroxyquinoline (antiseptic), benzethonium chloride (antiseptic), and other anti-infectives or microstatic agents.

Analgesics/NSAIDS can be added on a controlled-release basis through use of appropriate microstructures such as PolyPore E200, or they can be added directly. Potential analgesics/NSAIDS available for delivery with the wound sealant include acetylated/non-acetylated salicylates, ibuprofen, diclofenac, naprosyn, piroxicam, difunisal, oxaprozin, sulindac, tolmetin sodium, nabumetone, mefanamic acid, fulurbiprofen, fenoprofen, meloxicam, meclofenemate, etodolac, ketoprofen, diclonine, and indomethacin.

Local anesthetics can be added on a controlled-release basis through use of appropriate microstructures such as PolyPore E200 or they can be added directly. Potential local anesthetics available for delivery with the wound sealant include menthol, camphor, Lidocaine, Prilocaine, Benzocaine, Butacaine, Cyclomethycaine, Dibucaine, Tetracaine, Daspaicin, in addition to morphine and its derivatives.

Cell growth factors can be added on a controlled-release basis through use of appropriate microstructures such as PolyPore E200, or they can be added directly. Potential cell growth agents and factors available for delivery with the wound sealant include platelet-derived growth factor, granulocyte colony stimulating factor, fibroblast growth factor, and epidermal growth factor. These growth factors have been used for promoting wound healing by treating infected foot ulcers in diabetic patients, by treating pressure ulcers, and by treating venous leg ulceration.

Other potential actives available include ethyl chloride (vapocoolant), hydrocortisone, and phenylephrine (vasoconstrictor). These can be added on a controlled-release basis through use of appropriate microstructures such as PolyPore E200, or they can be added directly.

### Component F

### Blood Coagulation Components

In the embodiments containing clotting agents as Component F, the addition of clotting agents, such as human recombinant clotting components (some combination of thrombin, thromboplastin, or various other factors, cations, etc.), biochemically accelerates the thrombolytic cascade to produce a further improvement in the speed of clot formation and wound sealing. The clotting agents are admixed with or adsorbed to the surface of the nanoparticles of the binding agents (Component A) through hydrogen bonding. Subsequent reaction with more polar water from the wound site results in simple release of the adsorbed factors to allow ready solubility and subsequent reactivity.

A new |F| representing any one, or a combination of several, clotting agents including thrombolytic cascade accelerant(s), can be added as an additional |F| after the multi-component wound sealant has been admixed. The clotting agents are native derived or preferably recombinant thrombin and thromboplastin, prepared by any of several methods. The clotting agents can be dried or lyophilized in advance to form a grindable or dispersible powder; dried or lyophilized after addition to a non-aqueous formulation containing a defined percentage of a non-hydrogen binding liquid such as glycerol so as to form a grindable powder; dried by evaporation after addition to a non-aqueous, non-hydrogen binding solvent such as certain alcohols. The use of non-hydrogen binding materials avoids interactions between the silica nanoparticles in storage.

Preferably, the thrombolytic cascade accelerant is free of fibrinogen or fibrin-analog, and consists of thromboplastin and thrombin. These clotting factors activate cleavage of natural fibrinogen found at the wound site, which produce fibrin that leads to the desired thrombolytic cascade. The thromboplastin can be selected from a wide range of sources including simplastin, thromboplastin reagent, brain thromboplastin, British comparative thromboplastin, Thromborel S, calcium thromboplastin, porcine brain thromboplastin, ox brain thromboplastin, Innovin R, Recombiplastin, and others of similar characteristics. A preferred material is recombinant human thromboplastin. The thrombin (r-thrombin) is typically from activated recombinant human thromboplastin from human CHO cells using Hirudin and Hirudin-based peptide sepharose chromatography or produced by recombinant techniques known in the art.

Recombinant human thrombin and thromboplastin are available and are the reagents of choice for human use. The formulation is designed to be stable in both liquid and dry form, yet retaining and maintaining its specific reactivity and bioreactivity at peak levels. The formulation is also designed to maintain full functionality in the presence of the binding agents without interaction between the two components, or impediment of the hydrogen-bonding web formation by the binding agent. The wound sealant having clotting agents is formulated with and absorbed to the hydroxysilica nanoparticles. Alternatively or in addition, the clotting agents can be introduced as an admixture of low hydrogen-bonding polyfunctional short chain molecules, e.g., polyethylene glycol 3350, polyoxyethelene-6-sorbitol, or non-ionic surfactants such as polysorbate 60, in non-aqueous liquid form combined with thrombin and thromboplastin.

At the wound site, any weakly hydrogen-bonded thrombin or thromboplastin molecule coadsorbed to polyfunctional short-chain molecules or non-ionic surfactants immediately releases materials to hydrolysis upon primary hydration of the active silica nanoparticle carrier with the highly polar water available in the ambient body fluid. This results in the preferential binding of the hydroxyl groups on silicon dioxide (binding agent) to the more highly polar water molecules as the basis for web formation. This allows the clotting agents to be released into the fluid for accelerated clot formation, thus creating a one step, single-delivery, liquid admixture tissue sealant.

Fragments of clotting agents can be used as an alternative to using the whole polypeptide. Thrombin is not just an enzyme with moderately restricted proteolytic capabilities, yet extraordinarily high specificities for certain bonds (such as the A alpha-cleavage site in fibrinogen), but also is a protein with hormone-like activities involving cell receptor interactions. Such activities do not require the catalytically active enzyme, but are blocked by hirudin (also antithrombin III). These appear to involve a unique insertion and subsequent peptide segment at an axon junction. On the other hand, the enzymatic functions of thrombin depend on the catalytic site, per se, and derive specificity from the adjacent a polar-binding site within the fibrinopeptide side and the independent anionic-binding site within the fibrin side of the active groove. See, Fenton, J. W. et al., Thrombin active-site regions, Semin Thromb Hemost, 1986 July; 12(3):200-8, for a discussion of the specific thrombin peptide regions that are involved in the clotting pathway and are suitable thrombin peptide fragments for conjugation to binding agents as described herein. See also, McCallum et al., J. Biol. Chem., 1996 Nov. 8; 271(45):28168-75, for a discussion of specific thromboplastin peptide regions that are involved in the clotting pathway and are suitable thromboplastin peptide fragments for conjugation to hydroxysilica nanoparticles as described herein.

Additional clotting factors involved in clot formulation can be supplied as part the tissue sealant or simply provided by the body at the site, though they are not critical to effectiveness. They can be purified native (human or animal), or recombinant materials. Factors V, VII, and X can be additionally supplied for promoting the thromboplastin mediated reactions. In addition to this, or alternatively by itself, Factor XIII may be additionally supplied resulting in a thrombin-mediated clotting reaction. Likewise, various methods or improvements known in the art may be integrated or included in the wound sealant preparations disclosed herein.

As an example, the formulation described above can be modified to provide a liquid-stable thrombin through use of a polyol or other stabilizer (see for example, European Patent Application No. EPS 0277 096B1), addition of plasmin inhibitors (see also for example, U.S. Pat. No. 5,645,859), or inclusion of other blood clot techniques known in the art.

The clotting agents stimulate typical thrombin-like proteases supporting fibrinogen cleavage to fibrin. These permit the wound sealant's use in applications with heavy bleeding, trauma use, and applications of recurrent bleeding, even in cases of hemophilia, and even where the subject may be taking doses of blood thinners and anti-clotting agents. The two basic building blocks of the clot, namely prothrombin and fibrinogen, are supplied in relatively high levels by the body at the wound site. The clotting agents in the wound sealant preparation accelerate and catalyze the clotting process and use these naturally available clot proteins, the clotting effect working in parallel and tandem with the activated binding agent, which provides a matrix or lattice that traps blood cells and plasma for enhanced hemostasis.

The appropriate concentrations of thrombin and thromboplastin depend at least in part on whether the formulation is prepared for severe or more moderate bleeding. Generally enzyme concentrations per dose of a liquid dual-component wound sealant formulation range from 0.01 nanomolar to 10 micromolar of clotting agents, preferably 0.1 to 1000 nanomolar concentrations, more preferably 1 to 100-nanomolar concentration, and most preferably about 10 to 50-nanomolar concentrations of clotting agents. In a dry formulation, enzyme weights per dose of a powder/lyophilized dual-component wound sealant formulation range from about 1 nanogram to 100 mg of clotting agents, preferably 10 nanograms to 10 milligrams, more preferably 100 nanograms to 1 milligram clotting agents, and most preferably about 1 microgram to 100 micrograms of clotting agents. Modifications to the specific concentrations of each clotting agent are apparent to those of skill in the art, given published activities of the various clotting cascade enzymes at numerous concentrations. See, Lo K, Diamond S L, Blood Coagulation Kinetics: High Throughput Method for Real-Time Reaction Monitoring, Thromb Haemost., 2004 October; 92(4):874-82. Preferably, clotting agents do not saturate the silica nanoparticle surfaces; the hydrogen bonded lattice structure is desirable.

An appropriate dose of a wound sealant depends largely on the particular injury type, and can be assessed by a medical professional. Additionally, a subject with a clotting deficiency or disorder, or one taking blood thinner medications may require additional quantities of the appropriate formulation. By way of non-limiting example, a 2-cm laceration characterized by small-vessel bleeding may be treated using 1-500 mg or more of a powder formulation. A small puncture wound, e.g., from a needle or lancet stick may be treated using 1-10 or more mg of a powder formulation or 1 drop of a liquid formulation. Deep wounds may be packed with gram quantities of a sterile dry powder formulation, or with varying weights of single and dual-component dry formulations. Single component wound sealant preparations are predominantly silica and ceramic, and are generally inert in the body.

### Component G

### Activator/Accelerator Component(s)

Formulating an effective multi-component product |A|B|C|D|E|F| can require additional active factors to activate, accelerate, or balance the final formulated product. Such multiple-component mixtures can be put into different physical forms (powder, semi-gel, gel, or liquid). Examples of such components might be the addition of calcium cations to activate or accelerate the clotting process, or the addition of alkalizing or acidic constituents to adjust the pH of the final formulation. Each different MOM formulation can require different active components to optimally activate the final formulated wound sealant product. Since the amounts and comparative ratios of each |A|B|C|D|E|F|component are formulated to address a specific wound type and/or bleeding rate, more or less of a given activator or accelerator (|G|) may be required to bring the final formulation into balance for its intended use.

The activity of some Composition G components can be dependent upon pH. When necessary, the final pH of each product can be controlled using alkalizing or acidic constituents, or various physiological pH buffers and salts at appropriate strengths that are suitable for maintaining optimal pH conditions for the final product admixture.

Irradiation of the final product admixture may be used to prevent microbial contamination of the product. Azide, or other antimicrobial growth agent such as preparations containing elemental iodine (tincture of iodine—3% elemental iodine), or other preservatives such as Kathon, Neolone, or PVP-1 can also be used to prevent the final product admixture from being contaminated. Each of the admixture components can be treated separately with irradiation or azide or other preservative before formulating into the final admixture product. Sterile techniques can be used for preparing the final formulated product. Any changes in the properties of the final admixture that are caused by using irradiation and/or azide treatments are to be taken into considered when producing the final formulated product. Preferably, any procedure used to prevent microbial contamination would not diminish the safety, stability and/or effectiveness of the final product.

This invention provides a multiple-component wound sealant system with multiple functions, including cross-bridging of a binding agent to form a clot scaffolding, fluid (water) removal, adhesion and clumping upon hydration leading to bulk aggregation, thickening and swelling based on water removal, delivery of functional actives, and divalent cations inherent in the normal clotting cascade. To this, recombinant thrombolytic materials such as thromboplastin and prothrombin can be added. Coagulation and congealing can be readily achieved based on the materials used herein, regardless of the patient's own ability to achieve hemostasis, or the presence of medications or anticoagulants (heparin, for example) in the blood stream. The materials to be used depend on the size of the vessel (capillary vs. artery, for example) that is bleeding, as well as the wound type (scrape vs. trauma, for example).

Typical vessel closure involves pressure and/or natural clotting in and around the site of injury. Pressure constriction focused on closing off the capillary is one mechanism applicable if pressure is indeed used. Another mechanism based on this invention is for rapid capillary closure to stop leakage based on expansion of the additives. Since the materials added are micro and nano-particulate in nature and are added in vast excess from a mass and surface area perspective, they can serve to stop bleeding by rapid and controlled expansion at the site of injury. Expansion can promote intra-lumenal clogging to promote natural intra-lumenal clotting in addition to extra-lumenal pressure due to expansion and constant pressure. The pliability of the activated hemostat, coupled with the ability for the components to reorganize and reform after sheer, aids in control of sustained and recurrent bleeding under trauma conditions.

The present invention provides wound sealant compositions and methods to reduce, control, seal, or eliminate heavy bleeding from external vascular sites, internal vascular sites, and deep wound trauma. This invention has applications for controlling hemorrhage in various areas including: lacerations, burns, bedsores, intra-abdominal bleeding, heavy trauma, and intra-vessel bleeding. This invention also offers a novel solution to reduce death caused by uncontrolled internal or external hemorrhage in combat fighters.

The wound sealant products formulated from the MOM have applications in reducing bleeding from a wound site in a human. The wound sealants provide various methods of regulating hemostasis in a subject. Treatable wounds include: topical wounds; deeper wounds; surgical incisions; severe wounds; battlefield wounds and trauma; and emergency room excessive bleeding, among others. Accordingly, the various applications of the wound sealants include first aid and triage applications for surgical and medical procedures.

Cross-bridging binding agent |A| can be applied as a powder or as a coating, or blended with a non-aqueous low hydrogen bonding liquid or solvent at any concentration from under 0.1% to over 99.9%. It can also be blended with non-hydrogen-bonding materials such as aliphatic hydrocarbons (mineral oil) wherein other additives in the single-component |A| only, or the multi-component wound sealant formula |A| plus |B|C|D|E|F|G|, can be blended with, or coadsorbed to, the nanoparticles for ready delivery to the wound site. Upon contact with highly polar water within the fluid of the wound, the blended or coadsorbed materials are delivered to the fluid phase in exchange for nanoparticle hydrogen bonding to water molecules. This helps facilitate dispersion of wound sealant additives, such as antibiotics, analgesics, or other medications. The cross-bridging binding agent |A| can be delivered as a dry powder, or in a non-aqueous liquid carrier. It can be added to bandages as a non-aqueous gel, or as a powder. The binding agent can be admixed with a dry inert carrier such as talc, or a similar material, or coated onto or incorporated into any conventional wound dressing material.

To the silica nanoparticles in |A|, various soluble or insoluble, synthetic or naturally occurring short chain monomers or polymers can be added to the mixture in dry form as components G. Although not required for lattice formation, these |G| materials can be entrapped within the lattice itself further strengthening the web network in situ acting as a mordant (cement) between the cross-linked silica framework. Materials hereby incorporated by reference include but are not limited to: cross-linked anionic or cationic polyamine or polyacrylamide flocculent material (PAMS); lignosulfanates; hyaluronan; synthetic polyketides; polyhydroxyalkanoates, cutin or suberin digests of plant material (naturally occurring polyesters); poly(g-D-glutamate); polymerized human serum albumin (recombinant); bioplastic polymers like pullanan and the fungal polymer scleroglucan; and naturally occurring non-edible polysaccharides like dextran, and the like. Protein polymers including collagen and fibrinogen are also useful.

Fumed silica nanoparticles, as a single-component |A| only or as one of the multi-component agents, form a three dimensional lattice network within the fluid of a wound sample over a wide range of particle mass to fluid volume ratios. In the event of excess bleeding or excess fluid at a deep wound site, it can be advantageous to mix silica nanoparticle dry powder with other materials that can function as water absorbents. Water absorption functions as an activity secondary to lattice formation, facilitating the take up of fluid within the wound to aid the thickening of the sample and to facilitate clotting by enhancing the proximity of components, or to serve the opposite purpose of intentionally keeping the environmental interface of the wound hydrated (wet) to control the moisture loss rate, improve healing, and reduce scarring. Such properties may be especially useful in burn victims to control fluid loss rate. Owing to its high surface area, the silica nanoparticle powder is mixed with various conventional large particulate water adsorbent materials at varying ratios to facilitate wound fluid absorption. Such absorbents include ultra fine ground perlite (1600° C. heated silicaeous volcanic rock; 200-600% water absorption, % weight); ground heat expanded vermiculite (220-325% water absorption, % weight; 4-Superfine grade; 90-160 Kg/m3 density); cross-linked agarose gels such as Sephadex® and or Sepharose®; synthetic molecular sieve powders such as Purmol®; molecular sieve alumina, or alumina gels, or alumina silicate microspheres used in deodorants (Lawrence Laboratories; UOP); ceramic microspheres, zeolites, and/or inactive or activated carbon or charcoal. These materials at least double their weight with water and are compatible with the binding agents.

As a single-component single-delivery |A|-only system and as any one of the many multi-component/single-delivery |A|B|C|D|E|F|G| systems, the cross-bridging binding agent produces an immediate “freeze” effect upon blood flow due to its thixotropic effect upon the aqueous constituents of the blood and the creation of a web fabric that captures blood cells. The resulting clot consists of a synthetic wound dressing that supports continued clot formation. The fluid of the wound contains fibrinogen from the body, which meets the cross-linked web along with blood cells, platelets, and plasma, containing all other clotting factors ordinarily provided by bleeding, and collectively accelerates primary clot formation. The clot reforms as necessary at every level even with dimensions below one micron, maintaining coverage and sealing wounds and bleeding channels even at the capillary level.

The base scaffold structure is a cross-linked network of silica nanoparticles to which other components are added to modify the functional character of the final admixture wound sealant. Substances can be added to the admixture that don't impair the effectiveness of the admixture wound sealant for its basic purposes, but can deliver treatment to the wound site.

Antimicrobial agents can be delivered to the wound site. A preferred method involves adding the antimicrobial agent silver sulfadiazine and/or other such agents to the admixture to prevent bacterial and fungal contamination of the wound site. This prevents contamination of the wound site.

Oxygen can be delivered to the wound site. Hydrogen peroxide is a strong oxidizer and is highly germicidal. Catalase enzymes, which are found in most tissues exposed by injury, decompose hydrogen peroxide to generate oxygen. Hydrogen peroxide at the wound site provides both oxygen to aid in wound healing and a highly effective germicide.

A preferred approach involves adding hydrogen peroxide as an active component to the final multi-component nanoparticle admixture. Hydrogen peroxide becomes part of the final powder admixture and adds an effective oxygen releasing component to the final wound sealant product. At the wound site, wound fluids mix with the wound sealant to initiate the enzymatic conversion of hydrogen peroxide to oxygen. An oxygen rich atmosphere is created at the wound site.

Topical pain medication can be delivered to the wound site. The base scaffold structure is a network of silica nanoparticles to which other components are added to modify the character of the final admixture wound sealant. In addition, substances can be added to the admixture that don't modify the character of the final admixture wound sealant but can deliver various treatments to the wound site. For example, topical pain medication such as lidocaine, non-steroidal anti-inflammatory drugs (NSAIDs) such as ibuprofen, and other such agents are added to the admixture to help control the pain associated with the open wound.

The base scaffold structure provides a covering over the open wound site to provide protection from environmental contaminants and to provide a moist healing site.

The preferred base scaffold structure is a network of silica nanoparticles to which other components are added to modify the character of the final admixture wound sealant. In addition, substances can be added to the admixture that don't modify the character of the final admixture wound sealant but can deliver treatment to the wound site. As such, once delivered to the wound site, the wound sealant admixture interacts with blood, wound fluids and body constitutes to form a semi-gel covering over the wound site. This provides a protective barrier between the open wound and the outside environment. In addition, the semi-gel wound sealant provides a moist wound site covering that is conducive to wound healing.

Once the semi-gel wound sealant has formed a protective covering and a favorable wound healing environment, growth factors present in the wound sealant admixture are available to aid in wound healing. Also, adding chemical components that form oxygen in an aqueous environment can also be added. Also, local anesthetics and NSAIDs can be added to the wound sealant admixture to aid in controlling pain associated with the open wound. Also, antimicrobial agent silver sulfadiazine and/or other such agents can be added to the admixture to prevent bacterial and fungal contamination of the wound site. Various biological, genetic and cellular components can also be added to the admixture to be part of the final composition embedded inside the base scaffold structure for delivery to the wound site. The final multi-component admixture is formulated as a one-step single-delivery vehicle for application to the wound site.

The current invention provides a one-step multi-component nanoparticle admixture wound sealant to control bleeding. A preferred delivery of the admixture is by pouring the admixture onto the wound site as a powder or liquid formulation, and said dispensing can be by tare-and-pour, metered pouring, ejection from a container, etc.

A preferred embodiment is a powder hemostatic formulation for consumer (human) use, comprising a nanoparticle fumed silica cross-bridging binding agent, a fluid-removal agent, and a nonpolar liquid delivery agent. The formulation may be easily applied to the wound site in variable quantities, will quickly stop bleeding, and will adhere to the wound to provide a protective coating.

Another preferred embodiment is a powder hemostatic formulation for consumer (human) use comprising a nanoparticle fumed silica cross-bridging binding agent, a fluid-removal agent, a nonaqueous liquid delivery agent, and one or more of an adhesion/clumping agent, a thickening swelling agent, a calcium cation source, an anesthetic, an analgesic, an antiseptic, a vasoconstrictor, and a mild acid to control pH. The formulation may be easily applied to the wound site in variable quantities, will quickly stop bleeding, and will adhere to the wound to provide a protective coating.

Another preferred embodiment is a powder hemostatic formulation for animal use, consisting of a nanoparticle fumed silica cross-bridging binding agent, a fluid-removal agent, and a nonpolar liquid delivery agent. The formulation may be easily applied to the wound site in variable quantities, will quickly stop bleeding, and will adhere to the wound to provide a protective coating.

Another preferred embodiment is a powder hemostatic formulation for animal use, comprising a nanoparticle fumed silica cross-bridging binding agent, a fluid-removal agent, a nonaqueous liquid delivery agent, and one or more of an adhesion/clumping agent, a thickening swelling agent, a calcium cation source, an anesthetic, an analgesic, an antiseptic, a vasoconstrictor, a mild acid to control pH, and a bitter agent. The formulation may be easily applied to the wound site in variable quantities, will quickly stop bleeding, and will adhere to the wound to provide a protective coating.

Another preferred embodiment is to deliver using a multiple bag device. The admixture is contained inside a sealed biodegradable bag. The rapid biodegradable bag with the wound sealant admixture contained inside is placed inside a larger non-biodegradable bag. The larger outer bag can have a capacity of from 5 to 50 times the volume of the sealant contained inside the smaller biodegradable bag. The biodegradable inner bag rapidly dissolves upon contact with an aqueous environment. The outer bag does not dissolve upon contact with an aqueous environment, but permits rapid and easy transport of water, blood, and blood components through its wall. Blood and other aqueous tissue and blood components pass through the outer bag to dissolve the inner bag. This releases the wound sealant material to interact with blood, blood constitutes and wound tissue constituents to initiate and complete the clotting process. When clotting begins, the complex of nanoparticle wound hemostatic agent and clotted blood is contained inside the outer bag device. The outer bag is large enough to accommodate the thickening and swelling of both the clotting blood and the supporting nanoparticle scaffolding.

The two-bag device for administering the wound sealant makes it convenient for the hemostatic agent to be applied as a single-delivery vehicle. In addition, this two-bag device is convenient to remove from the site of injury and, if necessary, a second two-bag device administered to a wound that is still bleeding. Removing the first bag device removes blood clots, thereby offering immediate access to the wound site for the quick application of a second device. The size of the bag used can be varied and optimized to meet the needs of individual applications.

Another preferred embodiment is a powder hemostatic formulation for surgical or battlefield trauma use, comprising a nanoparticle fumed silica cross-bridging binding agent, a fluid-removal agent, and a thickening swelling agent. The formulation may be easily applied to the wound site as a one-step application in variable quantities, will quickly stop heavy bleeding, will quickly swell in the presence of wound fluid to effectively apply pressure to the wound site, and will be easily removed within hours to allow further treatment of the wound site by medical personnel.

Another preferred embodiment is a powder hemostatic formulation for surgical or battlefield trauma use, comprising a nanoparticle fumed silica cross-bridging binding agent, a fluid-removal agent, a thickening swelling agent, and one or more of a nonaqueous liquid delivery agent, an adhesion/clumping agent, a calcium cation source, an anesthetic, an analgesic, an antiseptic, a vasoconstrictor, and a mild acid to control pH. The formulation may be easily applied to the wound site as a one-step application in variable quantities, will quickly stop heavy bleeding, will quickly swell in the presence of wound fluid to effectively apply pressure to the wound site, and will be easily removed within hours to allow further treatment of the wound site by medical personnel.

The current invention may also be applied as a liquid, gel, semi-gel, aerosol, or fiber patch composite bandage, and dispensing can be by tare-and-pour, metered pouring, ejection from a container, spraying, pasting, etc

The carrier for preferred liquid, gel, semi-gel, or aerosol embodiments of the present invention would ideally be flexible and resistant to cracking, have no adverse effects on product stability, and could optionally aid in releasing therapeutic agents to the site of injury. Many such formulations will be clear to one skilled in the art, such as siloxane-containing polymers, cyanoacrylates, or pyroxylin, among others.

Pyroxylin solution is formed by mixing nitrocellulose in a suitable organic solvent such as diethyl ether or ethyl alcohol. The reaction takes place when activated with anhydrous acetic acid and in the presence of a suitable catalyst. The reaction product is precipitated in water or an alcohol. Quick-drying solvent-based pyroxylin solutions that contain nitrocellulose are used as a delivery liquid for over-the-counter wound care product formulations. The preferred method of applying quick-drying lacquers is by spraying or pasting to the site of injury and beyond. The nitrocellulose lacquer dries to produce a hard yet flexible, durable thin-film.

Suitable propellants include, for example, a chlorofluorocarbon (CFC), such as trichlorofluoromethane and 1,2-dichloro-1,1,2,2-tetrafluoroethane, a hydrochlorofluorocarbon, a hydrofluorocarbon (HFC), such as 1,1,1,2-tetrafluoroethane, 1,1,1,2,3,3,3-heptafluoropropane, carbon dioxide, dimethyl ether, butane, propane, or mixtures thereof. Preferably, the propellant includes a chloro fluorocarbon, a hydrochlorofluorocarbon, a hydrofluorocarbon, or mixtures thereof. More preferably a hydrofluorocarbon is used as the propellant. The propellant is preferably present in an amount sufficient to propel a plurality of doses of the wound care product from an aerosol canister.

Conventional aerosol canisters, such as those of aluminum, glass, stainless steel, or polyethylene terephthalate, can be used to contain the wound care product formulations according to the present invention. Aerosol canisters equipped with conventional valves, preferably, metered dose valves, can be used to deliver the formulations of the invention. The selection of the appropriate valve assembly typically depends on the components in the wound care formulation.

Liquid- or gel-based formulations are typically prepared as a two-phase mixture, i.e., one or more liquid and one or more solids that are admixed into a single product and stored as a solution or suspension. The primary solid component is a hydroxylated fumed silica nanoparticle powder, for example grade M-5P (Cabot), 0.1-99.9% wt/vol, or equivalent, preferably 1-20% wt/vol. An example of a preferred liquid phase is pyroxylin (collodion), a mixture of nitrocellulose with ether or acetone, sometimes also augmented with an alcohol. The solid phase is admixed into the liquid phase to form a non-aqueous evaporative solvent-based solution. Pyroxylin is a generic name for cellulose nitrate resin compounds that form a film when dissolved in a mixture of solvents like ether and alcohol. Other appropriate solvents may include acetone, ether, amyl acetate (Banana solution), alcohol (methyl alcohol, isopropyl alcohol, or ethyl alcohol), and others, as well as combinations of any of these materials. Various polymeric materials can be used in the liquid phase as well, such as cellulose nitrate (nitrocellulose), cellulose acetate, cellulose acetate butyrate, cellulose acetate proprionate, cellulose proprionate, ethyl cellulose, and carboxy methyl cellulose. Also, various non-cellulosic resins that can be used: poly(glycolic acid), poly(lactic acid), poly(e-caprolactone), poly(dioxanone), poly(hydroxybutyrate), poly(hydroxyvalerate), poly(dimethyl siloxane), poly(sebacic acid), poly(hexadecanoic acid), poly(ortho ester), poly(trimethylene carbonate), polymeric dextran, crosslinked polyamine or polyacrylamide flocculants, or any derivative or copolymer of the aforementioned materials. The liquid formulation can be sterilized by gamma irradiation or other medically acceptable liquid sterilization techniques.

The admixture is stirred and dispensed into a suitable container (plus lid) for consumer use, and may be applied by pasting, pouring, spraying, etc. When a thin film of liquid or gel hemostat is applied to the wound site, the solvent evaporates quickly, leaving a thin film covering the wound site. The hydroxyl groups on the silica dioxide nanoparticles are attracted to the more highly polar water molecules and to themselves in such a way that silica-solvent interactions are exchanged with water-silica interactions (hydrogen bonding) when the mixture contacts the wound site. This aids in solvent evaporation and enables the silica aggregates to form a lattice framework upon drying. The applied material covers and interacts with the wound site forming a clear plastic film over the wound site—a liquid bandage. This liquid bandage provides a protective covering and a lattice framework for clot formation that, in the example of pyroxylin-based formulations, will not wash off for several days. Three-dimensional cross-bonding of the silica upon solvent evaporation also facilitates wound closure serving as a nonaqueous based liquid suture.

In addition to the base hemostat formulation constituents discussed previously, clotting agents may be admixed with the liquid formulations to enhance performance of the hemostat. Clotting agents may interact with the silica nanoparticles through hydrogen bonding, or they may be adsorbed to excipients and mixed with silica nanoparticles into a liquid base. Thrombin or thromboplastin may be coadsorbed to polyfunctional short-chain molecules or non-ionic surfactants. At the wound site, the liquid bandage will immediately release clotting agents as they are exchanged with water molecules at the surface of the hydroxylated silica nanoparticles. Liquid dual-component wound sealant formulations include from 1 microgram to 1 milligram of clotting agent per 10 mg dry weight of silica nanoparticles, from 10 micrograms to 500 micrograms of clotting agent per 10 mg dry weight of silica nanoparticles, and from 100 micrograms to 250 micrograms of clotting agent per 10 mg dry weight of silica nanoparticles.

Various additives can also be combined with the liquid bandage formulation, for example antiseptic additives such as 8-hydroxyquinoline, Kathon, Neolone, alcohol, iodine, poly(vinyl pyrrolidone)/iodine, benzethonium chloride, and/or antibiotic additives such as polysporin, neosporin, penicillin, methicillin, cephalosporin, erythromycin, vancomycin, gentamycin, ciprofloxicin and other broad spectrum antibacterials, and/or antifungal additives such as terbinafine and amphotericin, and/or analgesic additives such as diclonine chloride, lidocaine, paracetamol, pramoxine HCl, ibuprofen, and or vasoconstrictors such as phenylephrine, epinephrine, thromboxane, and poly(n-acetyl glucosamine), and/or other absorbents and mordants additives as described above. These additives have also been found to work well in the dry (powder) formulations.

A preferred embodiment of the current invention provides a liquid hemostatic composition for topical delivery on minor abrasions, cuts, scrapes, scratches, burns, sunburns, ulcers, internal venous bleeding, external venous bleeding, and surgical trauma, with said composition comprising of a nanoparticle fumed silica cross-bridging binding agent and a nonaqueous liquid carrier for forming a thin-film barrier over the site of injury. The formulation may be easily applied to the wound site in variable quantities and will quickly stop bleeding.

Another preferred embodiment of the invention provides a liquid hemostatic composition for topical delivery on minor abrasions, cuts, scrapes, scratches, burns, sunburns, ulcers, internal venous bleeding, external venous bleeding, and surgical trauma, with said composition comprising of a nanoparticle fumed silica cross-bridging binding agent, a nonaqueous liquid carrier for forming a thin-film barrier over the site of injury, and one or more of a fluid-removal agent, a thickening swelling agent, an adhesion clumping agent, a calcium cation source, an anesthetic, an analgesic, an antiseptic, a vasoconstrictor, and a mild acid to control pH. The formulation may be easily applied to the wound site in variable quantities and will quickly stop bleeding.

Another preferred embodiment of the invention provides a liquid hemostatic composition for topical delivery on minor abrasions, cuts, scrapes, scratches, burns, sunburns, ulcers, internal venous bleeding, external venous bleeding, and surgical trauma, with said composition comprising of nanoparticle fumed silica cross-bridging/binding agent, at least one local anesthetic agent, and a nonaqueous liquid carrier for forming a thin-film barrier over the site of injury, and for promoting and prolonging contact of the anesthetic agent with the site of injury.

Another preferred invention provides a topical delivery method for prolonging analgesia on minor abrasions, cuts, scrapes, scratches, burns, sunburns, ulcers and other skin injuries and irritations with said method comprising the step of applying topically to site of injury a composition comprising at least one local anesthetic agent and a carrier for forming a long-lasting thin-film or other barrier over the site of injury and for promoting and prolonging contact of the anesthetic agent with the site of injury.

Another preferred invention provides the use of a composition for topical delivery comprising at least one local topical anesthetic agent and a nonaqueous liquid carrier in the preparation of a topical medicament for providing prolonged analgesia to the site of injury wherein said carrier forms a long-lasting thin-film or other barrier over the site of injury and prolongs contact of the anesthetic agent with minor abrasions, cuts, scrapes, scratches, burns, sunburns, ulcers and other skin injuries and irritations at the site of injury.

Another preferred invention provides the use of a liquid hemostatic composition for topical delivery comprising at least one local topical anesthetic agent and a carrier in the preparation of a nonaqueous liquid topical medicament for providing prolonged analgesia to a subject having minor abrasions, cuts, scrapes, scratches, burns, sunburns, ulcers, external venous bleeding, internal venous bleeding, or surgical trauma, wherein said liquid composition forms a long-lasting thin-film or other barrier over the site of injury and prolongs contact of the anesthetic agent at the wound site.

Another preferred invention provides the use of a gel hemostatic composition for topical delivery comprising at least one local topical anesthetic agent and a carrier in the preparation of a liquid topical medicament for providing prolonged analgesia to a subject having minor abrasions, cuts, scrapes, scratches, burns, sunburns, ulcers, external venous bleeding, internal venous bleeding, or surgical trauma, wherein said gel composition forms a long-lasting thin-film or other barrier over the site of injury and prolongs contact of the anesthetic agent at the wound site.

Another preferred invention provides the use of a hemostatic semi-gel composition for topical delivery comprising at least one local topical anesthetic agent and a carrier in the preparation of a liquid topical medicament for providing prolonged analgesia to a subject having minor abrasions, cuts, scrapes, scratches, burns, sunburns, ulcers, external venous bleeding, internal venous bleeding, or surgical trauma, wherein said gel composition forms a long-lasting thin-film or other barrier over the site of injury and prolongs contact of the anesthetic agent at the wound site.

Another preferred invention provides the use of a hemostatic aerosol composition for topical delivery comprising at least one local topical anesthetic agent and a carrier in the preparation of a liquid topical medicament for providing prolonged analgesia to a subject having minor abrasions, cuts, scrapes, scratches, burns, sunburns, ulcers, external venous bleeding, internal venous bleeding, or surgical trauma, wherein said gel composition forms a long-lasting thin-film or other barrier over the site of injury and prolongs contact of the anesthetic agent at the wound site.

Another preferred invention provides the use of a hemostatic patch composite bandage composition for topical delivery comprising at least one local topical anesthetic agent and a carrier in the preparation of a liquid topical medicament for providing prolonged analgesia to a subject having minor abrasions, cuts, scrapes, scratches, burns, sunburns, ulcers, external venous bleeding, internal venous bleeding, or surgical trauma, wherein said fiber patch composite bandage composition forms a long-lasting thin-film or other barrier over the site of injury and prolongs contact of the anesthetic agent at the wound site.

The preferred invention provides topical medicament agent compositions that promote wound healing when used on minor abrasions, cuts, scrapes, scratches, burns, sunburns, ulcers, external venous bleeding, internal venous bleeding, or surgical trauma, with said composition comprising at least one topical wound medicament agent and a carrier for forming a thin-film or other barrier over the site of injury, and for promoting and prolonging contact of the anti-microbial agent with the site of injury.

### EXEMPLIFICATION

### Introduction

A goal in severe trauma management is to immediately stop and control blood loss. After cessation of primary bleeding from a single or multiple body sites and stabilization of critical vital functions to avoid exsanguination, the patient oftentimes requires aftercare treatments for primary wounds. This usually involves surgical intervention for vessel closure and tissue repair. Avoidance of exsanguination due to excessive blood loss is the primary goal of the traumatologist. Post trauma, wounds must be left in a condition that allows for ready surgical access and/or wounds must be properly closed, treated, and managed in such a way as to promote optimal wound healing.

The care provided to trauma injury varies depending on the severity and type of injury, on the location and environment where the trauma occurred, and on the type of assistance available. Excessive bleeding during heavy physical trauma at an accident scene or by a soldier from a battlefield wound in an unclean environment requires fast and immediate action to stop the bleeding from multiple body sites to avoid administration of factor VIII and the adverse sequalae from its use. It is critical to stabilize the patient for proper follow-up care. Some products currently being used by the United States Armed Forces include QuikClot (Z-Medica) and HemCon Chitosan Bandage (HemCon). With surgical trauma, wound closure and wound healing care are usually provided in the controlled, safe environment of a surgical suite and/or hospital. With bedsores, where the wounds have failed to produce anatomic and functional integrity, primary treatment is provided by managing the wound-healing process over time.

New techniques, devices, and drugs for bleeding and bleeding and/or hemorrhage control are being developed and applied across the continuum of trauma care: pre-hospital, emergency room and operative and post-operative critical care. Despite all of the technology currently available to treat trauma patients, bleeding and hemorrhage control is still the major unresolved problem in emergency medical care. Approximately 50% of all deaths in the first 48 hours of hospitalization are related to an inability to control bleeding. Failure to stop bleeding within the first 24 hours is almost uniformly fatal especially when multiple trauma sites are involved. Unfortunately, the methods currently being utilized to stop otherwise fatal hemorrhage are hundreds of years old.

In military combat, as well as civilian trauma, immediate action is highly effective in limiting patient mortality, since most bleeding fatalities occur within the first 30 minutes of the injury. It is generally accepted that hemostatic products for forward care in the battle zone must control bleeding quickly, be ready to use, simple to apply, have a shelf life approaching two years and prevent bacterial or viral transmission. The product's hemostatic action is time-critical to meet both military and civilian needs.

Devices being investigated or used today as external methods of wound intervention include absorbent pads containing clotting agents, topically-applied clotting or bleeding cessation agents in powder or granule form, pressure bandages, gauze, tourniquets for extremities, and trauma kits for wounds to the body.

Agents designed to stop external bleeding differ in composition and ingredients and help the rapid formation of a clot at the site of application. Clotting products generally contain high concentrations of materials such as human fibrinogen, thrombin, calcium, factor XIII and anti-fibrinolytics.

An external hemostatic control bandage, developed jointly by the U.S. Army and the Red Cross, uses fibrin to mimic the final stages of blood coagulation. The components used in this fibrin bandage are naturally occurring clotting agents and work by presenting fibrin hemostatic clotting agents faster and in higher concentration than the body does. This leads to faster clot formation. In addition to fibrin, microporous polysaccharide macrobeads, mineral and synthetic zeolites, and a shellfish derivative usually referred to as chitosan (poly-N-acetyl glucosamine) are also available for use in controlling hemorrhage.

A number of new hemostatic products are available for treating wound trauma. A bandage product using chitosan (deacetylated poly-N-acetyl glucosamine base, HemCon Inc., Tigard, Oreg.) is being used by U.S. troops. Unfortunately, it has a shelf life of only 18 months and its cost is prohibitive for routine use.

Z-Medica Corporation, Wallingford, Conn., provides a pressure bandage product (QuikClot) for use by U.S. troops. QuikClot uses a granular, synthetic mineral zeolite (proprietary formula of zeolite volcanic mineral granules) to stop bleeding by adsorbing liquid and promoting clotting. However, because of the materials used, QuikClot generates heat that can cause burns if the bandage isn't applied correctly. Use of QuikClot is further complicated because it can take too much time to get the clotting material out of the wound once the injured gets to a hospital area.

ActSys Medical Inc., Westlake Village, Calif., provides a hemostatic gauze product, ActCel, that meets two key battlefield functionalities: simplicity and speed. The ActCel product is a collagen-like natural substance created from chemically treated cellulose. It expands when in contact with blood to sealing off damaged vessels and aid clotting and is registered with the Food and Drug Administration (FDA) to help control bleeding from open wounds and in body cavities. This hemostatic gauze expands to 3-4 times its original size when in contact with blood. In the hospital area, the clotting materials can be easily washed away.

Medafor Inc., Minneapolis, Minn., uses a bioinert, microporous polysaccharide macrobead product that is synthesized from potatoes (TraumaDEX). This powdered microporous polymer product stops bleeding by expanding at the wound site and dehydrating the blood. The body absorbs the material within 48 hours. At present, TraumaDEX is being developed for operating room indications.

Another non-bandage approach employs a non-zeolite topical powder containing a hydrophilic polymer and potassium salt (Quick Relief, Sarasota, Fla.). Quick Relief states that a flexible, protective scab quickly forms to cover the wound site when the powder contacts the blood and slight pressure is applied. The produce is available for sale to government buyers for the harsher environment of warfare. The product is sold primarily for nose bleeds.

Another class of devices works only under pressure and excludes hemostatic agents. First Care Products Inc., an Israeli company, has brought a combat compression dressing to the field. This Emergency Bandage, also known as the “Israeli Bandage,” was introduced as an upgrade to first aid products for use by the military.

Cinch Tight universal compression bandage developed by H&H Associates, Bena, Va., is a baseline combat first aid product, which is used by the U.S. Marine Corps. Cinch Tight is designed to control arterial bleeding. This bandage can be deployed using only one hand and features an 8-inch by 10-inch absorbent pad, an S-hook and Velcro strips for quick attachment. It can be applied as a sling, as a bandage for chest wounds or a compression dressing for abdominal wounds. It operates both as a compression bandage and as a tourniquet.

No perfect solution currently exists for treating excessive bleeding. Heat generation with respect to one type of agent is a major problem. The dressing's ability to adhere effectively when applied to deep wounds or wounds of irregular shape creates another major limitation. The ability to deal with excessive blood is another limitation, as is treatment and control of pressure bleeding from arterial bleeding. Sometimes cleansing a hemostatic agent from the wound can be a problem.

### Surgical and Trauma Wounds

Surgical and trauma wounds are the most common types of wounds addressed in the wound-care area. There are millions of surgical procedures performed annually worldwide; in the United States alone, there are over 100,000 surgeries performed daily. Some surgical procedures are performed following stabilization after initial stabilization of vital functions and cessation of bleeding.

Current bandages are made of gauze and are often applied in conjunction with an elastic bandage. They allow the wound to breath but are not good barriers to subsequent contamination. These bandages lack antimicrobial properties and cannot stop serious bleeding and require the application of pressure in the case of arterial bleeding. In addition, the ability of these classic methods to stop bleeding is largely dependent on the subject's individual ability to clot, and can be compromised if the subject has been administered a blood thinning medication or an anticoagulant. There is a need for an improved product that is antimicrobial, that is resistant to subsequent infection and contamination, and that can still stop massive hemorrhage. A bandage that can protect either multiple major wounds or large surface area wounds from subsequent contamination and at the same time reduce pain and infection is also needed.

Superficial wounds currently are closed primarily with sutures. But suturing requires a moderate level of training by the health care provider. Some wounds following trauma are not closed immediately until the patient stabilizes as further surgical intervention is required for vessel closure. Closing wounds using cyanoacrylate glues have received regulatory approval for limited use on small closures, but are not used routinely for external wound closure.

Conventional wound sealants fail to present an optimized combination of speed of clotting, effectiveness under pressure bleeding conditions, and clots that are dynamic over time in response to the needs of the trauma site. Typical wound sealants are usually used in conjunction with separate wound dressings. Sometimes the wound sealants are applied to the fluid surface above and away from the clot itself as an attempt to glaze or seal over the wound.

Scarring in skin after trauma, surgery, or burn injury often results in adverse aesthetics, loss of function, restriction of tissue movement and/or growth and adverse psychological effects. Current treatments of pressure garments, silicone dressings, hydrocortisone injections, etc. are empirical, unreliable, and unpredictable. There are no prescription drugs for the prevention or treatment of dermal scarring. However, recent studies have identified therapeutic targets for preventing scarring. By altering the ratio of growth factors present during adult wound healing, wounds heal perfectly with no scars, at an accelerated healing rate, and with no adverse impact on wound strength or wound infection rates. The opportunity exists for the topical delivery of the proper ratio of growth factors for improved wound healing without scars. Use of artificial skin or cadaver skin can contribute to infections, result in tissue rejection, and contribute to scarring.

Clearly, surgical trauma caused by sharp objects occurs in a clean or sterile controlled environment where wound closure is optimal. However, trauma wounds not caused in a controlled environment are often intermediate sized, widespread, and dirty wounds with considerable tissue damage such as in traffic accidents or on the battlefield.

Not all battlefield wound trauma is immediately life threatening due to excessive bleeding. Other types of wound trauma that occur routinely on the battlefield still need to be treated. Abrasions are generally caused by scraping of the skin's outer layer; incisions are cuts commonly caused by knives or other sharp objects; lacerations are jagged, irregular cuts or tears of the skin; punctures are caused by an object piercing the skin layers, creating a small hole; and burns cause damage to skin cells that may vary greatly in depth, size, and severity. Wounds due to firearms can be deep and massive with substantial tissue destruction. Dismemberment due to trauma requires immediate intervention to stop blood loss from the severed limb. Any wound may involve venous and arterial bleeding, the latter involving pressure bleeding. Proper care of each type of wound requires eventual adherence to the principles of cleanliness, wound covering, tissue apposition, and protection from physical trauma while tissues return to their normal physiological state apply to all wounds.

### Trauma Pain

Pain from severe trauma is interpreted by nerve endings (nociceptors). Severe trauma pain can cause the autonomic nervous system to respond by immobilizing the body to defend against additional injury, raising respiratory rates, releasing hormones such as epinephrine to help minimize pain, and increasing blood pressure and heart rate to ensure that vital organs receive adequate blood flow. Severe pain can also cause the brain to release natural painkillers (e.g., endorphins, enkephalins) to help minimize trauma pain in certain circumstances.

Minimizing trauma pain in individuals with sudden, acute injuries is an important step to their recovery. Currently, severe pain is controlled by morphine. This drug completely incapacitates the patient who is now no longer able to help with his or her own care. Morphine also depresses respiration and heart rate and is dangerous with some injuries and lethal if it is not administered properly. As a result, acute trauma can often lead to inadequate pain treatment. Pain control is important to the patient since isolating and treating trauma pain 1) minimizes discomfort, 2) results in shorter recovery times, 3) creates fewer complications, and 4) lowers mortality rates.

Some would argue that local anesthetics are underutilized in the trauma setting. With new delivery methods and low toxicity, these agents are seeing more widespread use in managing chronic pain. However using local anesthetics to treat severe acute trauma pain is infrequent at best, especially for primary intervention.

Local anesthetics such as lidocaine are used to block pain sensation from specific areas of the body by blocking nerve impulse conduction. Non-steroidal anti-inflammatory drugs (NSAIDs) such as diclonine, diclofenac, and ibuprofen are widely used analgesic and anti-inflammatory drugs for treating pain.

The present invention relates to topically delivering pharmaceutically suitable local anesthetic and NSAID drugs to areas of acute trauma to provide pain control. A preferred local anesthetic is lidocaine. Diclonine is a preferred NSAID. The local anesthetic and NSAID drugs have suitable solubility, stability and therapeutic characteristics in aqueous environments to be effective for the topical treatment of localized wound pain such as surgical pain, burn pain, open wound pain, ulcer pain, and severe trauma pain.

### Topical Wound Healing Constituents

Devices being investigated or used today as external methods of wound intervention include absorbent pads containing clotting agents, topically-applied clotting or bleeding cessation agents in powder or granule form, pressure bandages, gauze, tourniquets for extremities, and trauma kits for wounds to the body. Agents to stop external bleeding are designed to help the rapid formation of a clot at the site of application. Clotting products generally contain high concentrations of materials such as human fibrinogen, thrombin, calcium, factor XIII and anti-fibrinolytics.

### Burn Injury

Approximately 27 million burn cases requiring treatment occur worldwide each year and of those 7 million will require hospitalization. More than a million will die as a direct result of their burns. Burn injuries are particularly difficult and painful area of medicine requiring multi-pronged approach to address infection, pain and a host of long-term complications. Continued advances in biotechnology have driven the burn portion of the wound-care industry, estimated at over $1.5 billion globally. However, advances in treating initial burn shock, infection control, early wound closure, and modulation of the hyper metabolic response, have decreased morbidity and mortality.

Physiologic alterations resulting from burn injury can be minimized by adequately maintaining tissue perfusion, early excision of burn wounds, and rapid wound coverage. These measures in combination with antibiotic coverage will decrease the hyper metabolic response and the incidence of sepsis that can lead to hemodynamic instability and organ failure. Anabolic agents such as recombinant human growth factor and pharmacologic agents that modulate inflammatory and endocrine mediators (e.g., ibuprofen and propranolol) are used to treat severe burn injuries.

Wound healing for burn victims involves a complex process of cellular interactions. The most promising wound growth factors for modulating healing include epidermal growth factor, fibroblastic growth factor, platelet-derived growth factor and transforming growth factor-alpha. Receptors for these factors are expressed by many cells found in a wound. Topical application of epidermal growth factor demonstrated accelerated wound healing. Addition of a combination of these wound growth factors can improve wound healing of burn injuries.

In massive thermal injuries in which autologous donor skin is limited, wound closure must be achieved through alternative techniques. Major advances have been made with wound dressings and skin substitutes. These include allogenic skin, noncellular matrix material with a silastic covering membrane to mimic the physical properties of the epidermis, and epidermal cell culture techniques. Unfortunately these techniques have not been optimized and have resulted in high failure rates, copious scar formation, and tenuous skin coverage—all at exorbitant costs.

Sepsis is the major cause of death among burn patients. However because of early wound excision, topical antimicrobials and improved wound dressings, the incidence of such infections has decreased significantly. The development of topical antimicrobial agents, the use of perioperative system antibiotics and wound surveillance techniques has lead to decrease in mortality rates due to wound sepsis. The topical antimicrobial agent often used for burns is silver sulfadiazine, a sulfa medicine that prevents and treats bacterial or fungus infections. The general use of oral and topical antifungal agents has been shown to decrease candidal infection.

Traditional burn wound management involved applying topical antibiotics in dressings, which are changed twice daily until the eschar separated in from 3 to 5 weeks, and then directly applying topical antibiotics. Separation occurred by liquefaction of necrotic burn tissue by proteolytic enzymes released from proliferating pathogens within the wound. However, it has been shown that early removal of the burn tissue by tangential excision surgery reduced pain, decreased the number of operative procedures and shortened the length of hospital stay. Patients achieved better functional and aesthetic results. In addition because the original tissue was removed before vascular granulation tissue was formed, blood loss and mortality rate decreased.

New wound-care products and devices are entering the market and include improved synthetic dressing materials, xenogeneic tissue scaffold, bilayered human dermal substitutes, recombinant growth factors, and other techniques and materials. In addition to product innovations, disease management programs that emphasize prevention and early intervention appear to be effective.

### Decubitus Ulcer, Pressure Ulcers, and Chronic Skin Wounds

Chronic skin wounds are a major source of morbidity, lead to considerable disability and are associated with increased mortality. The incidence of chronic wounds in the U.S. is 5 to 7 million per year. Chronic wounds can lead to complications such as infections, contractures, depression or limb amputations. A lack of effective treatment options for the wound-care industry adds rising costs of skin ulcer treatment. New products are sought that will improve healing rates and prevent wound formation.

Skin wound healing occurs in three phases: 1) the inflammatory phase where neutrophils and macrophages enter the wound site, 2) the proliferative phase where tissue regeneration is supported by an increase of fibroblasts and endothelial cells, and 3) the remodeling phase where skin replaces scar tissue.

However, chronic wounds do not progress to healing. Skin wound disorders are heterogeneous and complex with a variety of causes. Most are classified as pressure ulcers, diabetic ulcers, arterial insufficiency vascular ulcers, venous insufficiency vascular ulcers or burns.

Wound healing is characterized by numerous growth factors acting by stimulating chemotaxis, cellular proliferation, extracellular matrix formation, and angiogenesis with contraction and reestablishment of cellular integrity. The efficacy of growth factors in enhancing wound healing has been demonstrated both in vivo and in vitro. In addition, wound care such as debridement, callus reduction, and control of infection is important in promoting wound healing. Debridement enabled removal of necrotic tissue, drainage to be maintained, and better surface contact with human epidermal growth factor (EGF). Callus reduction also helped to reduce excessive pressure on the wound. Antibiotics are prescribed upon clinical suspicion of infection or positive culture results. Human EGF at 0.04% (wt/wt) when topically applied was successful in reducing the healing time.

Common current treatments for standard care of skin wounds include debridement of necrotic or infected tissue, maintenance of a moist wound environment, control of infection, and nutritional support. Wound-specific additional treatments are employed for pressure ulcers, diabetic ulcers, vascular ulcers and burns.

New potential methods for treating skin wounds include topical growth factors, bioengineered skin products, electrical stimulation, therapeutic ultrasound, novel dressings (e.g., hydrocolloids, alginates), hyperbaric oxygen, and gene therapy. Other techniques include vacuum-assisted closure and low-level laser therapy.

Dressings that promote a moist environment to assist healing have been used in recent years. It was also found that re-epithelialization proceeded 1.5 times more rapidly if the wound was occluded. Occlusive dressings have been clinically successful on patients with chronic wounds, and they reduce pain and improve convenience. Advances in dressing technology have not yet results in the development of materials that correct abnormalities in the healing cascade, with the exception of dressings containing hyaluronic acid that specifically promotes healing.

### Topical Growth Factors

Wound repair involves inflammation, induction of tissue factor, formation of a fibrin matrix, and growth of new smooth muscle vessels. This process involves a complex interaction between cells, mediators, growth factors, and cytokines. The cascade of events starts with activation of the procoagulant pathway and recruitment of inflammatory cells and is followed by a phase of cellular proliferation and tissue repair of the injury. Tissue factor is the major initiator of the extrinsic coagulation cascade, is involved in all phases of the host response to wounding, and is likely playing a central role in wound healing.

Clinical results from topical application of growth factors to chronic wounds have not been dramatic. Only platelet-derived growth factor has been approved for treating non-infected foot ulcers up to 5 cm² in diabetic patients. Growth factor has also added some value in treating pressure ulcers. Granulocyte colony stimulating factor, fibroblast growth factor, and epidermal growth factor have been used in clinical ulcer trials. Growth factors administered at intervals that more closely mimic the normal healing process may provide more promising results. The diversity of growth factors and types of chronic wound suggest that these factors have potential as new treatments if patients' individual requirements can be identified.

### Bioengineered Skin Equivalents

For successful management of pressure ulcers, both cutaneous and subcutaneous tissues need to be grafted, particularly over boney prominences.

Large epidermal sheets from cultured cells obtained by skin biopsy have been used to treat patients with extensive burns, but with only limited success. However, the potential benefit of this technology led to the development of skin equivalents. Commercial products include a dermal matrix without immunogenic cells, such as ALLODERM, commercially available from Lifecell Corp. of Branchburg, N.J. and a combination of dermal fibroblasts and bovine collagen, such as INTEGRA, commercially available from Integra Life Sciences Corp. of Plainsboro, N.J. Living dermal replacement tissue, such as DERMAGRAFT, commercially available from Advanced Biohealing, Inc. of New York, N.Y., consists of non-immunogenic neonatal fibroblast cultured on a polyglactin mesh and has been used to treat burns and diabetic foot ulcers and venous leg ulcers. All of these products have been used to treat wounds.

Bioengineered skin replacements are absorbed into the wound bed and are believed to exert their effect on chronic ulcers, at least in part, by altering the profile of cytokines within the chronic wound, thought the exact mode of action is unknown. Current skin replacement products include Epidermal (cultured Autologous epidermal cells) for severe burn injuries, Dermal, ALLODERM and INTEGRA FDA approved for treating burns, Dermagraft-TC, commercially available from Advanced Tissue Sciences Inc. of La Jolla Calif. and Apligraft, commercially available from Sandoz AG of Switzerland, for treating diabetic foot ulceration, and composite culture skin (collagen matrix with fibroblasts and epidermal cells). The human fibroblasts are seeded onto a bioabsorbable polyglactin mesh scaffold and proliferate to fill the interstices of the scaffold. In the process, the fibroblasts secrete human dermal collagen, growth factors, and cytokines to create a three dimensional dermal substrate. Over time, the donor cells are replaced by the patient's own cells of which none contain DNA of the graft. Improved wound healing rates have been reported.

Future opportunities for treating chronic wound trauma includes gene therapy, which may allow genes important in healing to be delivered directly into a wound and vascular endothelial growth factor, which may be an important component in the promotion of angiogenesis. The real challenge for the future is to select appropriate interventions for each patient.

### Topical Oxygen Treatment

The ability to oxygenate tissue is compromised in many skin sores, ulcers, wounds and burns. Poor oxygen delivery can cause slow healing, infections, scar development, and even tissue death and amputation. Tens of thousands of patients die each year in the U.S. as a result of complications from insufficient delivery of oxygen to compromised tissue.

In wounds of large surface area such as ulcers, only the tissue at the edges or base of the ulcer is well supplied with blood. The growing granulation tissue must be supplied by diffusion from blood vessels and plasma, a relatively inefficient process. Both systemic and topical oxygen have been used to treat these skin diseases.

In topical hyperbaric oxygen therapy, oxygen is applied directly to an open wound. The oxygen dissolves in tissue fluids and improves the oxygen content of the intercellular fluids. Skin disorders treated with topical hyperbaric oxygen include osteomyelitis, burns and scalds, necrotizing fascitis, pyoderma gangrenosum, refractory ulcers, diabetic foot ulcers, and decubitus ulcers. Cuts, abrasions and surgically induced wounds or incisions may also benefit from topical oxygen therapy.

The healing of surface wounds and burns is improved by increasing the wound oxygen tension using an oxygen-generating wound dressing using chemically generated oxygen. The wound dressing is capable of supplying oxygen through chemical reaction using immobilized solid hydrogen peroxide and a decomposition catalyst. The oxygen-generating dressing is applied over a hydrogel occlusive wound covering.

### Minor Abrasions and Lacerations—Consumer Applications

There remains a need for improving consumer treatment of minor cuts, scrapes, burns and the like. In the past, the health care industry's focus has been on providing a bandage and skin protectant for infection prevention and pain reduction that is simple to apply, flexes during movement, and adheres better than conventional adhesive bandages. For wounds or applications that are not superficial, tissue sealant and skin closure applications have received considerable attention.

Powders, liquids, lotions, creams, and pastes that remain wet or dry and form a film or crust are well known in the industry. Examples include zeolite- or polysaccharide-based powders, tincture of benzoin (gum), collodion, modified ethyl acetate, cellulose nitrate, cyanoacrylate, and pyroxylin in solutions. Some trade names include “Urgent QR”, “QuikClot”, “Liquid Bandage”, “Skin Shield”, and “Nu-Skin”. U.S. Pat. No. 4,880,416 issued to Horichi et al. described a dermal bandage comprising a film like adhesive for protecting wounds. U.S. Pat. No. 4,584,192 issued to Dell et al. described a film forming compositions for protecting wounds and releasing anti-microbial agents to the skin. U.S. Pat. No. 4,156,067 issued to Gould described a polyurethane polymer that could be used in drug delivery systems or as burn dressings. Each of these materials and patents provide skin protection from external contamination, some with added medicinal properties, but few have demonstrated the ability to stop bleeding of a wound quickly and adequately or aid in retention of lost blood, and some have adverse side-effects such as strong exothermic reactions or sting to the wound site. In addition, all have high coefficients of friction that increase the likelihood of abrasion or irritation of the skin surface.

Various examples of liquid compositions can be found in the prior art. U.S. Pat. No. 6,183,593 issued to Narang et al. describes an adhesive composition for treating wounds in the body joint areas, e.g., elbow, which comprises polydimethylsiloxane and polymerizable 1,1-disubstituted ethylene adhesive monomer, U.S. Pat. No. 4,987,893 issued to Salamone et al. describes liquid coating compounds forming conformable bandages comprising siloxane-containing preferred additional polymer, volatile poly(dimethyl-siloxane) and optimal polar liquid. U.S. Pat. No. 6,605,667 issued to Badejo et al. describes an adhesive composition for medical purposes, e.g., retarding blood flow from wounds, comprising polymerizable 1,1-disubstituted ethylene monomer, and antioxidant stabilizer, e.g., pentamethyl chromanol and/or non-phenolic antioxidant. U.S. Pat. No. 5,259,835 issued to Clark et al. describes a wound closure device for surgical incisions, lacerations, etc. that comprises a porous bonding member placed across a wound that receives flowable liquid adhesive through openings in the top of the member.

Liquid compositions have also been proposed for delivering various therapeutic agents for treating wounds. U.S. Pat. No. 6,143,805 issued to Hickey et al. describes a sterilization of liquid adhesives in a container. WIPO Patent No, WO 06/096914 issued to Sheil et al. describes a composition, useful to treat significant open wounds, comprising a local anesthetic agent and a carrier for forming a long-lasting barrier over the open wound and for promoting and prolonging contact of the anesthetic agent with the wound. U.S. Pat. No. 7,071,166 issued to Nishida et al. describes skin wound healing promoters or skin epidermal extension promoters containing substance P analogs and insulin-like growth factor-I for treating wounds like tears, abrasions, surgical incisions, skin ulcers, or burns. U.S. Pat. No. 6,391,323 issued to Carnevali describes a topical composition for treating burns, sunburn, abrasions, ulcers and cutaneous irritation. U.S. Pat. No. 6,383,502 issued to Dunshee et al. describes a coating composition for application to skin as sunscreen, e.g., comprising a siloxane containing polymer, alkane based siloxy polymer reaction solvent, and adjuvants. US Patent Application No. 20060240083 issued to Klein et al. describes a medical composition useful as a topical composition for application to wounds and surgical sites comprising a beta glucan compound and elemental silver or silver compound.

Liquid compositions have also been used to protect wounds by forming a film that covers the area of injury. U.S. Pat. No. 6,942,683 issued to Dunshee describes a wound closure system that applies flowable adhesive skin paint that includes a wound bridging portion of microporous polypropylene film. The film retains the opposing edges of wound together by adhering wound to skin on opposing sides of wound. U.S. Pat. No. 6,627,216 issued to Brandt et al. describes a spray-on fluid composition used for drug delivery and bandage formation comprising a tacky component, a film-forming non tacky component, and a volatile solvent. U.S. Pat. No. 6,512,023 issued to Malofsky et al. describes stabilized monomer adhesive compositions with improved shelf life. U.S. Pat. No. 5,981,621 issued to Clark et al. describes a polymerizable monomer-type tissue adhesive for wound closure that contains a cyanoacrylate, a plasticizing agent, and an acidic stabilizer, and forms a flexible and strong bond in or bridging the wound site. U.S. Pat. No. 5,103,812 issued to Salamone et al. describes a conformable, room temperature film-forming bandage or coating compound comprising a siloxane-containing terpolymer, a liquid poly(dimethyl-siloxane) and an optimal polar liquid. U.S. Pat. No. 6,958,154 issued to Brandt et al. describes a spray-on fluid composition used for drug delivery and bandage formation comprising a tacky component, a film-forming non-tacky component, and a volatile solvent. U.S. Pat. No. 6,646,119 issued to Tanaka et al. describes the manufacture of an acetylated nitrocellulose coating material that involves distributing nitrocellulose in a dispersion medium, followed by solid phase acetylation, U.S. Pat. No. 5,126,123 issued to Johnson describes an aerosol drug inhalation formulation that contains 1,1,1,2-tetrafluoroethane propellant and a soluble surfactant. US Patent Application No. 20060030808 issued to Kennedy describes a fluid composition for forming a protective bandage for superficial minor cuts, abrasions, burns and wounds, comprising a cyanoacrylate monomer formulation containing a preset amount of a specific fluid monomer and a fluid cyanoacrylate monomer component.

Currently available internal tissue sealant products are fibrin-based, protein-based, and/or synthetic-based. Surgical hemostats products include thrombin-based hemostats, oxidized regenerated cellulose-based hemostats, gelatin-based hemostats, collagen-based hemostats, and autologous-based hemostats. Acute wound closures include sutures, staples, suture-less closures using radiofrequency energy devices, and surgical zippers using multilayered adhesives. Biomaterials targeted for treating skin lacerations include various bioabsorbable products.

Each biomaterial is well suited for certain wound sealant uses. However, none provides an optimal environment for wound healing. Most available biomaterials are used as temporary wound coverings that are later removed to allow the body to heal itself. Preferably, biomaterials for wound care cover and protect acute wounds, as well as set the stage for accelerated healing. Biomaterials that provide a microenvironment suitable for and conducive to angiogenesis and cellular proliferation and differentiation are desired. None of these products is a true one-step formulation that delivers the multiple functions desired by medical professionals in an ideal wound closing and healing agent.

### Pain and Infections in Minor Lacerations and Abrasions

Pain from minor wounds is initiated from traumatized nerve fibers and can lead to prolonged hypersensitivity of surrounding tissues and significant discomfort. Such wounds are usually treated by covering and closing the site of injury by bandaging or other practice. Closing the wound will stop any minor bleeding and protects the traumatized tissues and nerve fibers from dehydration, ongoing environmental exposure, risk of infection and ongoing painful stimulation. Pain therefore abates as the inflammatory response and tissue edema subsides.

Pain related to such wounds is managed using systemic analgesia (such as oral, IM or IV opioids or non-steroidal anti-inflammatory agents) and injected local anesthetic agents when short term wound anesthesia is required. In addition, local anesthetic agents block nerve conduction to reduce or eliminate pain sensation for from 30 minutes to several hours depending on the agent and method of administration.

Some local anesthetic agents may be applied topically and typically provide anesthesia for 30 to 60 minutes in open wound situations. Slow-release vehicles may double the 30 to 60 minutes of analgesia. Also, prolonged analgesia may be achieved using anesthetic agents with long duration of action, such as bupivacaine (6-8 hours duration of action), or repeated subcutaneous injection or combining an anesthetic agent with a slow-release vehicle such as was described in US Patent Application No. 2003/0185873.

Generally there is also a need for an antimicrobial medical composition for use with or as part of a topical composition. The antimicrobial medical composition may be further adapted for use as a wound dressing and/or as a component of topical preparations that protect minor abrasions, cuts, scrapes, scratches, burns, sunburns, ulcers and other skin injuries and irritations from further environmental insult and deliver analgesic, antiseptic, and skin healing promoting activity to the wound.

Antimicrobial and immunostimulating agents are known components of topical compositions, wound dressings, and surgical meshes. Examples of these uses are given in published U.S. Pat. Application No. 20060240083 and issued U.S. Pat. No. 5,980,918 to Klein et al. and issued U.S. Pat. No. 5,676,967 to Williams et al. There is a need with respect to all topical compositions, wound dressings and surgical meshes to provide an effective antimicrobial function.

### Wound Healing

General treatment of minor abrasions, cuts, scrapes, scratches, burns, sunburns, ulcers and other skin injuries and irritations is to give first aid to site of injury and wait for the spontaneous recovery of the injury due to natural restoration properties of a living body. However, such spontaneous recovery can require extended periods of time until restoration is complete and the associated pains gone. It is therefore desirable to promote wound healing positively by administering therapeutic agents that promote wound healing to the site of injury.

New epithelial tissues and connective tissues are formed by migration and proliferation of cells in a healing process of minor abrasions, cuts, scrapes, scratches, burns, sunburns, ulcers and other skin injuries and irritations. Drugs that promote or stimulate migration, differentiation and proliferation of cells participates in wound healing and can be therapeutic agents for wounds.

Powder hemostats and liquid bandage formulations are available to the Over-the-Counter (OTC) consumer market. Liquid bandage preparations are ideal for covering and protecting minor lacerations and abrasions, friction blisters, hangnails, finger cracks, and paper cuts. When applied to the skin the pyroxylin-based solution evaporates to form a plastic protective film over the application area and to promote healing. The polymerized film covering creates a moist wound healing environment to increase the rates of wound epithelialization and wound healing compared with conventional dressings. Most liquid bandage preparations claim to stop minor bleeding, create a protective seal over the wound, and keep out water, dirt and germs. These preparations generally act as a mechanical barrier to common microbial organisms and other forms of contamination. Powder-based hemostats generally claim to stop bleeding of minor lacerations and abrasions. They act by forming a crust, or artificial scab, over the wound site, and can take several minutes to be effective.

Liquid bandage products are available from numerous commercial sources and include New Skin Liquid Bandage, Curad Spray Bandage, Nexcare Bandages Spray Liquid Bandage, Liquid Bandage by J&J, and Skin Shield Liquid Bandage. Powder-based hemostats are also widely available OTC in products such as QuikClot (Z-Medica), Urgent QR and Nosebleed QR (BIOLIFE), TraumaDEX and Bleed-X (Medafor), Celox (MEDTRADE Biopolymers), ActCel (ActSys Medical), and Quick Relief.

Various materials and methods are used as examples for treating various wound trauma applications including cuts, scrapes, burns, bedsores, intra-abdominal bleeding, heavy surgical or battlefield trauma, and intra-vessel trauma.

At least one method for preparing a specific product for a specific wound application includes a three-step process:

Step One: Determine the optimal product features that are needed based on the type of wound and the primary wound treatment objectives.
Step Two: Using a matrix of materials, build an admixture of components that optimally addresses the primary wound treatment objectives.
Step Three: Formulate an admixture of components designed in Step Two into a one-step single-delivery final product.

### Example 1 Wound Treatment for Burns

### Formula 1

### Two Step Process

This formulation contains two components; first, Waterlock A180 (GPC) is applied as a powder directly on the burn area to form a hydrogel layer upon absorption of serous fluid. This takes approximately 30 minutes to complete. The formulation can be reapplied as necessary. Next, ATS (Engelhard) ceramic sorbant powder is applied to form a moisture holding barrier on top of the hydrogel and to form a “dry-to-the-touch” layer on the exterior of the hydrogel.

### Formula 2

### One-Step Process

In this one-step process, two components are mixed. Waterlock A180/ATS sorbant are mixed in a 80/20 w/w ratio. The powder formulation is applied directly on the burn area to form a hydrogel layer upon absorption of serous fluid.

Additives to Formula 1 and Formula 2

Several components can be added to Formulas 1 and 2 to improve the wound sealant performance. Components include the cross binding agent HS-5 from Cabot Corporation (1-5%), the delivery agent Poly Pore E200 (1-5%) from AMCOL used to deliver an antibiotic or silver, humectant agents ATS Ceramic Particle from Engelhard, Polyhydric Alcohol, and others, and osmotic pressure additives that includes buffers of appropriate osmolarity and ionic strength such as sodium phosphate buffer at 0.15 ionic strength and pH=7.4 (i.e., matching the buffering conditions of typical human blood), salts such as NaCl (at physiological concentration), acids or bases to adjust pH of the formulation, such as NaOH or ascorbic acid, protein stabilizers such as human serum albumin (HSA) or casein, and alcohols such as ethanol, isopropanol, glycerol, polyvinyl alcohol, or sugars such as galactose, maltose, etc.

### Example 2 Wound Treatment for Bedsores

### Unhealed Open Wounds with Serous Exudate

### Formula 1

A wound-healing product suitable for treating bed sores can be formulated from ATS (Engelhard) ceramic sorbant powder, sprinkled on the bedsore, and the excess removed. The sorbant absorbs the serous fluid, remains “dry to the touch”, aids in scab formation, and holds moisture to keep underlying wound areas hydrated. The covering remains pliable. The ATS ceramic sorbant powder can be removed and reapplied daily or as needed. The ceramic sorbant contributes to the formation of a scab and to the healing of the wound. The powder is not absorbed by the body and is for external use only.

### Formula 2

The wound-healing product can be formulated from a mixture of ATS sorbant (Engelhard)/EH-5 (Cabot) silica nanoparticle in an 80/20 ratio. Combining the sorbant (Formula 1) with the EH-5 cross bridging agent provides structural integrity and further promotes scab formation, imbues resistance against abrasion, and promotes rapid healing. This Formula 2 is not absorbed by the body and is for external use only.

Additives to Above Two Formulas

Additional components can be added to either of the previous two formulas to enhance the effectiveness of the wound sealant. An exemplary formula consists of the cross binding agent HS-5 at 1-5% (Cabot Corporation), a component to deliver an antibiotic such as Poly Pore E200 (AMCOL), a humectant such as ATS Ceramic Particle (Engelhard) or a polyhydric alcohol, and other additives such as buffers of appropriate osmolarity and ionic strength, salts at physiological concentrations, protein stabilizers such as HSA or casein, and alcohols such as ethanol, isopropyl alcohol, glycerol, polyvinyl alcohol or sugars such as galactose, maltose, and others.

### Example 3 Intra-Abdominal/Brain “Gel” Formulation

### Formulation 1

### Firm Gel

An exemplary firm gel wound sealant is formulated using a 61/33/6 w/w ratio of the following powders, respectively: Waterlock G400 (GPC); ATS sorbant Waterlock A220 (GPC); Aerosil COK 84 (Degussa). Applied directly to the wound site, the powder formulation will absorb wound exudates and swell into a film, cohesive gel.

### Formulation 2

### Soft Gel

An exemplary soft gel wound sealant is formulated using a 64/30/6 w/w powder ratio of Waterlock G400 (GPC), poly(acrylic acid) (Aldrich) or Waterlock A180 (GPC), and Aerosil COK 84 (Degussa), respectively. Applied directly to the wound site, the powder formulation will absorb wound exudates and swell into a soft, cohesive gel.

### Example 4 Heavy Bleeding Trauma/Stabilization Mordant

### Formula 1

### Hard Packing

Hard packing formulations are designed to provide wound sealant product that is fast clumping, sets quickly, is highly expandable, is highly absorbable, and is extra-firm in its hardness. The formulated admixture includes May 25, 1943/25/2 w/w ratio of powders EH-5 (Cabot), ATS Sorbent (Englehard), SUSPENGEL 325 PLUS, WATERLOCK G 220 (GPC) or POLARGEL Volclay NF-BC (ACC) and POLYPORE E 200 (AMCOL). Another formulation uses an alternate ratio of the same components: Oct. 15, 1960/13/2.

### Formula 2

### Soft Packing

Soft packing formulations are designed to set up quickly with minimal clumping, be moderately expandable, highly absorbent, and provide a soft and pliable wound sealant. The formulated admixture includes 10/23/15/50/2 w/w ratio of powders EH-5 (Cabot) or COX 84 (Degussa), ATS sorbant (Engelhard), Panther Creek 200 (ACC) or CAL-BEN 200 (Cimber) or Hectalite GM (ACC), Waterlock G 400 (GPC), and POLYPORE E 200 (AMCOL).

### Example 5 Vessel Injectible/Expandable Formulation

The wound sealant properties of this formulation include solid pellets of varying diameter and length for vessel insertion by catheterization. These pellets are held in place in the vessel lumen with catheter tip until expanded and bleeding stops (1-2 min swell time). The pellets are used to stop vessel leakage caused by trauma (especially brain). This product has use in treating aneurysm. The solid pellets are highly expandable for pressure-based closure of a vessel and are kept dry within a catheter until inserted. The pellets are non-dissolvable and non-leaching, and can be removed by surgery with subsequent vessel reconnection. Finally, the pellets are effective regardless of the subject's blood type, or the presence of blood thinners or anticoagulants such as sodium heparin, in the blood stream.

One example of the pelletized form is formulated from an admixture Oct. 4, 1970/15/1:w/w ratio of powders EH-5 (Cabot), ATS (Englehard), Suspengel 325 (Cimbar) or Hectalite GM (ACC), Waterlock G 400 (GPC) or Polargel Volclay NF-BC (AMCOL), and PolyPore E 200 (AMCOL).

Additional features are provided by the multiple component formulation. EH-5 adds three-dimensional structural integrity to the solid form. ATS sorbent aids in drawing fluid into the pellet, resulting in pellet expansion. Suspengel expands and clumps when wet. Waterlock adds plasticity and limited pliability to the pellet and acts as a binding and swelling agent as well. Polypore is available for drug delivery. Makall Silica Gel highly absorbent products are also available as binding and swelling agents as well as for drug delivery.

The forms and sizes of the solid pellets are to be determined by the final swell ratio, by the inner diameter of the blood vessel, and by the intra-vessel pressure due to blood pressure. The solid pellet forms can be encased in quickly-dissolving gelatin coat to aid handling. The solid pellet forms can be irradiated to sterilize.

### Example 6 Effectiveness of Nanoparticle Wound Sealant Containing Silver Sulfadiazine

### (A)

This wound sealant is formulated using a 75/15/5/5 w/w ratio of the following powders: Waterlock G400 (GPC), ATS sorbant (Engelhard), EH-5 silica nanoparticles (Cabot) or Degussa equivalent and PolyPore E200 (Amcol). In some embodiments, silver sulfadiazine is added to the admixture.

The effectiveness of nanoparticle wound sealant containing silver sulfadiazine to suppress bacterial growth in simulated burn wounds are evaluated by comparing with Silvadene, a commercial cream preparation of silver sulfadiazine. Comparisons are performed using an approved preclinical animal model protocol for assessing wound treatments in rats. Bacterial counts are followed for at least 7 days and the overall bacterial counts compared between the various groups: control, Silvadene cream, and nanoparticle wound sealant.

### (B)

This wound sealant is formulated using a 75/15/5/5 w/w ratio of the following powders: Waterlock G400 (GPC), ATS sorbant (Engelhard), EH-5 silica nanoparticles (Cabot) or Degussa equivalent and PolyPore E200 (Ameol). In some embodiments, silver sulfadiazine is added to the admixture.

In this study the dosing frequency of nanoparticle wound sealant with silver sulfadiazine is compared with the daily administration of 1% silver sulfadiazine cream to assess the influence of dose frequency on the antibacterial efficacy. This study shows that the nanoparticle wound sealant with silver sulfadiazine provided at least an equivalent antibiotic efficacy as the Silvadene cream alone, but with much fewer changes than current daily requirement for Silvadene cream.

### Example 7 Hydrogen Peroxide as a Source for Oxygen in Wound Sealant

This wound sealant is formulated using a 75/15/5/5 w/w ratio of the following powders: Waterlock G400 (GPC), ATS sorbant (Engelhard), EH-5 silica nanoparticles (Cabot) or Degussa equivalent and PolyPore E200 (Amcol). Hydrogen peroxide is complexed by forming a final product of hydrogen peroxide complexed in the final admixture at 10 percent or more by weight. Sample is dried at elevated temperature to form a stable final admixture containing hydrogen peroxide.

### Example 8

### Liquid Bandage Formulations

### (A)

An exemplary liquid band formulation is prepared by mixing a 1% solution of powder formulated using a 75/15/5/5 w/w ratio of the following powders: Waterlock G400 (GPC), ATS sorbant (Engelhard), EH-5 silica nanoparticles (Cabot) or Degussa equivalent and PolyPore E200 (Amcol) with a pyroxylin-based solution. When applied as a thin film of the final solution to skin, the pyroxylin-based solution evaporates to form a plastic protective film over the application area.

### (B)

Another exemplary liquid bandage formulation is prepared by mixing 1-5% (wt vol) of EH-5 fumed silica and 10% (vol/vol) D,L-lactic acid (Sigma) in Collodion (Malliknckrodt). When applied as a thin film of the final solution to skin, the pyroxylin-based solution evaporates to form a plastic protective film over the application area. Similar solutions may also be prepared using the above ratios while omitting the D,L-lactic acid component.

### (C)

The admixture in (A) can also contain other components to aid in preventing infection, reducing pain and promoting healing.

To determine the efficacy of the formulations described above, an adult volunteer study was conducted. All participants were apparently healthy normal adults with no history of bleeding disorders and no use of blood thinning agents. A small needle prick was made using a lancet in two duplicate spots and gently expressed to induce uniform minor bleeding at the wound site as would occur upon puncture or alternatively a raspy file was dragged across the skin to abrade it to induce minor bleeding as would occur upon abrasion. Care was taken to generate comparably sized cuts.

Immediately after puncture or abrasion, dry powder without thrombolytic factors was sprinkled generously onto one of the two cut sites. Excess powder was shaken off after 45 seconds and relative clotting time, relative scab tightness, and uniformity after 24 hours, and relative duration of the scab till it fell off were recorded.

### Example 9 Dry Powder Hemostats for Minor Cuts, Abrasions, and Lacerations

### (A)

An exemplary powder-based hemostatic formulation is prepared by thoroughly mixing a 80/20 (w/w) ratio of HS-5 fumed silica (Cabot) and ATS sorbent powder (Engelhard), respectively.

### (B)

An exemplary powder-based hemostatic formulation is prepared by thoroughly mixing a 63/16/20 (w/w) ratio of ATS sorbent powder (Engelhard), HS-5 fumed silica (Cabot), and glycerol (Fisher) or ethyl alcohol (Aldrich), respectively.

### (C)

Another exemplary powder-based hemostatic formulation is prepared by combining a 42/42/10/4/2 (w/w) ratio of ATS sorbent powder (Engelhard), HS-5 filmed silica (Cabot), glycerol (Fisher) or ethyl alcohol (Aldrich), L-Ascorbic Acid (Fisher), and Calcium L-Ascorbate dehydrate (Aldrich), respectively.

Another exemplary powder-based hemostatic is prepared by thoroughly mixing a 75/25 (w/w) ratio of HS-5 fumed silica (Cabot) and CBV712 sorbent powder (GSA Resources), respectively.

Numerous examples are available demonstrating the efficacy of the above exemplary formulations to enable hemostasis and clot-formation.

The formulation described in powder hemostat (A) was applied to a volunteer adult with a severe laceration abrasion combination on the forearm over a 3 square centimeter area. The wounds were bleeding steadily when the hemostat was applied with light pressure. The normal coagulation time for the volunteer without treatment was 11 minutes on average. The individual was on 325 mg per day aspirin and 75 mg per day Plavix for 2 years prior to testing. Within 45 seconds to a minute the wound had stopped bleeding, and a solid clot was formed at each of the multiple wound sites tested. The hemostat remained as a solid protective coating for several days, withstanding normal bathing behaviors of the subject. Re-bleeding was not observed throughout, and the wound healed normally with minimal scarring.

The formulation described in powder hemostat (A) was applied to another volunteer adult over a continuous three year period for all naturally occurring light to moderate lacerations, abrasions, puncture wounds, needle pricks, etc. that were incurred during that time period. The individual is diabetic and was on aspirin (325 mg) and sodium warfarin anticoagulant therapy on varying doses up to 10 mg per day for 5 years prior to treatment and for the three years of treatment in addition to use of several different anticoagulants such as Lepirudin to treat acute ischemic stroke in the latter half of the treatment phase. Normal clotting times for this individual prior to treatment ranged from 4 to >12 minutes depending upon the site and nature of the wound. Fingerstick bleeding, for example, from daily glucose testing generally took 5 minutes to stop. For all cases of injury recorded during treatment over three years, the individual was able to stop bleeding in <1 minute on average for all wounds without exception. Application also involved treatment of several severe lacerations to the hand incurred from work activities during the treatment phase.

The formulation described in powder hemostat (B) was applied to a series of anticoagulated blood samples from multiple species and multiple donors, including human, porcine, equine, canine, feline, and rat. All samples had been treated with sodium EDTA upon collection, and refrigerated until being heated to 37° C. just prior to experimentation. In all species, the powder hemostat forced clotting of the blood samples within 30 seconds, and did not result in any measurable hemolysis. Furthermore, the hemostat was shown to be efficacious regardless of species, donor, human ABO/Rh subtype, or the type of anticoagulant tested (sodium heparin, sodium EDTA, and sodium citrate were all tried). Finally, these studies demonstrate the power of the powder formulation in achieving hemostasis independent of fibrin, even in a donor treated with anticoagulants, and in the absence of applied pressure.

The formulation described in powder hemostat formulation (B) was also tested against various powder hemostats currently available on the market, including Urgent QR and Nosebleed QR (BIOLIFE), Bleed-X (Medafor), Yunan Baiyao (a well-known Chinese panacea), and Kwik-Stop Stypic Powder (Rich Health, intended for companion animal use). 0.35 g of each hemostat was applied to 0.25 mL of porcine blood (treated with sodium heparin and warmed to 37° C.) on a 45 mm piece of nitrocellulose filter paper (0.22 μm). Samples were evaluated 1 minute after application. While the powder hemostat described in Example 8 quickly absorbed the blood sample and formed a solid, firm, clot, Urgent QR, Nosebleed QR, Bleed-X, and Kwik-Stop had only formed a crust over the surface of the blood droplet, and did not appear to have absorbed any excess blood. The Yunan Baiyao sample was completely unchanged. Disturbance of the clots formed from all samples except the powder formulation describe in Example 8 resulted in gushing of the unclotted blood out from under the formed crust.

The formulation described in powder hemostat (C) was applied to a bleeding dermal incision on the back of a rat, to the right of the spine. The powder was applied directly into the wound site, without applied pressure. Hemostasis was achieved within seconds, and a solid, cohesive clot was formed. An identical wound was created on the back of the rat, to the left of its spine, and was allowed to bleed until hemostasis was achieved naturally. Bleeding persisted for several minutes, followed by congealing of the blood and finally clot formation after 10 minutes. The edges of the untreated wound were tightened and distorted, while the edges of the powder-treated wound remained undistorted in any way. This example demonstrates the potential for the powder hemostat to not only achieve hemostasis quickly, but to reduce scar formation as well.

The non-biological materials described herein direct blood clotting in the presence of anticoagulant independent of the host's thrombolytic cascade. This demonstrates the hemostatic power of the technology.

From the foregoing detailed description of the invention, it should be apparent that unique methods and compositions for inducing blood coagulation have been described resulting in improved therapeutic use. Although particular embodiments of the invention have been disclosed herein in detail, this has been done by way of example for purposes of illustration only, and is not intended to be limiting with respect to the scope of the appended claims which follow. In particular, it is contemplated by the inventor that substitutions, alterations, and modifications can be made to the invention without departing from the spirit and scope of the invention as defined by the claims.

What is claimed is:

1. A wound management composition comprising: a preparation of silica dioxide particles of less than about 200 nm, and a particulate surface area up to about 500 square meters per gram, wherein the composition has an average of four hydroxyl groups per square nanometer.

2. The composition of claim 1, further comprising a fluid removal agent selected from the group consisting of: ceramics, alumina or alumina silicate, silica or alumina gel, ceramic sorbent powder cationic exchanger, synthetic zeolyte Y powder, cross-linked polyamine, polyDADMAC, polyacrylamide, sodium polyacrylic acid, lignosulfates, silicaeous perlite, vermiculite, porous non-activated or activated carbon and hyaluron.

3. The composition of claim 1, further comprising an adhesion and/or clumping agent selected from the group consisting of: starch copolymers, poly 2-propenamide-co-2-propenoic acid including sodium or potassium salts thereof, bentonite clay, sodium or calcium bentonite, montmorillonite clay, smectite clay and magnesium lithium phyllosilicate.

4. The composition of claim 1, further comprising a thickening and/or swelling agent selected from the group consisting of: smectite clay, montmorillonite clay, sodium and calcium bentonite powder, aluminum oxide, magnesium aluminum silicates and nanosilica, sodium polyacrylic acid, and starch copolymers, poly 2-propenamide-co-2-propenoic acid including sodium or potassium salts thereof.

5. The composition of claim 1, further comprising a continuous release drug or therapeutic delivery agent selected from the group consisting of: allyl methacrylate cross polymers, cross-linked agarose gels, and natural or synthetic drug delivery vehicles.

6. The method of claim 5, wherein the drug is selected from the group consisting of: anti-infectives, analgesics, astringents, anti-inflammatory agents.

7. The composition of claim 1, further comprising an anti-infective agent selected from the group consisting of: silver sulfadiazine, neomycin triple antibiotic, methicillin, vancomycin, silver nitrate (silver ions), 8-hydroxyquinoline, Kathon, Neolone, PVP-Iodine, and other anti-infectives, microstatic agents, or anti-septics.

8. The composition of claim 1, further comprising an analgesic agent selected from the group consisting of: acetylated/non-acetylated salicylates, ibuprofen, diclofenac, naprosyn, piroxicam, difunisal, oxaprozin, sulindac, tolmetin sodium, nabumetone, mefanamic acid, fulurbiprofen, fenoprofen, meloxicam, meclofenemate, etodolac, ketoprofen, indomethacin, menthol, camphor, ethyl chloride, lidocaine, prilocaine, benzocaine, butacaine, cyclomethycaine, dibucaine, tetracaine, daspaicin, opioid analgesics and morphine and its derivatives.

9. The composition of claim 1, further comprising a cytokine selected from the group consisting of: platelet derived growth factor, granulocyte colony stimulating factor, fibroblast growth factor and epidermal growth factor.

10. The composition of claim 1, further comprising a thrombolytic cascade accelerant selected from the group consisting of: polyethylene glycol 3350, polyoxyethelene-6-sorbitol, non-ionic surfactants, polysorbate 60, polypeptide clotting factors, prothrombin, thrombin, thromboplastin and active fragments thereof.

11. The composition of claim 10, wherein the clotting factors are recombinant polypeptides.

12. The composition of claim 11, further comprising a mordant selected from the group consisting of: cross-linked anionic or cationic polyamine or polyacrylamide flocculent material (PAMS), lignosulfanates, hyaluronan, synthetic polyketides, polyhydroxyalkanoates, cutin or suberin digests of plant material, naturally occurring polyesters, poly(g-D-glutarate), polymerized human serum albumin, bioplastic polymers, pullanan, scleroglucan, naturally occurring non-edible polysaccharides, dextran, polypeptide polymers, collagen, fibrinogen, guar gums, xanthum gums, cellulose, carboxy methyl cellulose, soluble or insoluble fiber, alginate, agar, agarose and starch.

13. The wound management composition of claim 1, further comprising an oxygen source.

14. The wound management composition of claim 1, in a formulation selected from the group consisting of: a liquid, a coating on a bandage or patch, a foam, an aerosol, a gel and a semi-gel.

15. The wound management composition of claim 1, wherein the silica dioxide particles have a diameter of about 10 nm.

16. A method of making a wound sealant composition, comprising:
a. obtaining a preparation of silicon dioxide particles having a size less than 200 nm, wherein the particulate surface area is up to about 500 square meters per gram,
b. hydroxylating the silicon dioxide particles to an average of four hydroxyl groups per square nanometer; and
c. forming an admixture of the hydroxylated silicon dioxide particles with one or more agents selected from the group consisting of: a fluid removal agent, an adhesion and clumping agent, a thickening and swelling agent, a drug or therapeutic delivery agent, an anti-infective agent; an analgesic agent, a thrombolytic cascade accelerant, a mordant, monovalent or divalent cations and an oxygen source,
thereby forming a wound sealant composition.

17. The method of claim 16, further comprising admixing the wound sealant composition with an excipient, a surfactant, or a resin.

18. The method of claim 16, further comprising conjugating a polypeptide clotting agent or fragment thereof, to the hydroxylated silicon dioxide particles.

19. A method of treating a wound comprising identifying a subject having a wound characterized by excessive bleeding, and administering to the subject a wound sealant composition of claim 1, thereby reducing or ameliorating the excessive bleeding.

20. The method of claim 19, wherein the wound is an acute wound, a surgical wounds/incision, a traumatic wound, a penetration wound, a laceration, an abrasion, a contusion, a dismembered limb, a pressure ulcer, a venous insufficiency ulcer, an arterial ulcer, a neuropathic ulcer, a diabetic ulcer, and a burn wound.

21. A method for inducing blood coagulation in a subject, comprising administering a hemostatic formulation comprising fumed silica.

22. The method of claim 24, wherein the hemostatic formulation is a powder.

23. The method of claim 24, wherein the subject is treated with one or more anticoagulants.

24. The method of claim 27, wherein the one or more anticoagulants is aspirin.

25. A wound binding agent comprising reactive submicron colloidal silica particles that agglomerate in the form of a supra-molecular, cross-linked network that form a base scaffolding component as the structural basis for a synthetic non-fibrin clot.
